(19) United States
(12) Patent Application Publication
(10) Pub. No.: US 2003/0234468 A1
(43) Pub. Date: Dec. 25, 2003

(54) Soft, absorbent material for use in absorbent articles and process for making the material

(76) Inventors: Krishnakumar Rangachari, Savannah, GA (US); Erol Tan, CG Zutphen (NL); Steven Scott Harlen, Jesup, GA (US); Kimberly W. Sumner, St. Simons Island, GA (US); David S. Varney, Odum, GA (US)

Correspondence Address: WOOD, PHILLIPS, KATZ, CLARK & MORTIMER, 500 W. MADISON STREET, SUITE 3800, CHICAGO, IL, 60661, US

(21) Appl. No.: 10/396,305

(22) Filed: Mar. 25, 2003

Related U.S. Application Data
(63) Continuation of application No. 09/633,019, filed on Aug. 4, 2000, now abandoned.
Application No. 09/633,019 is a continuation-in-part of application No. 09/390,018, filed on Sep. 3, 1999, now Pat. No. 6,485,667.
Application No. 09/390,018 is a continuation-in-part of application No. 09/341,340, filed on Jul. 8, 1999, now abandoned.
Application No. 09/341,340 is a continuation-in-part of application No. 08/948,987, filed on Oct. 10, 1997, now Pat. No. 5,916,670.
Application No. 08/948,987 is a continuation-in-part of application No. 08/784,536, filed on Jan. 17, 1997, now Pat. No. 5,866,242.

(51) Int. Cl. A61F 13/15; D04H 1/70

(52) U.S. Cl. 264/122; 264/510; 264/518; 264/112; 264/121

(57) ABSTRACT

An absorbent material is provided with improved characteristics. A process is provided for making the absorbent material. A web is formed with at least one layer of absorbent material, and it may include a mixture of cellulosic fibers and superabsorbent material. The moisture content of the web is increased so as to increase the web density. Then, the web is compacted at an elevated temperature to further increase the web density and preferably to also effect hydrogen bonding within the web. In one embodiment a tissue layer is provided, is wetted with water, and is bonded to the layer of absorbent material.

### CROSS-REFERENCE TO RELATED APPLICATIONS

This application is a continuation of the U.S. patent application Ser. No. 09/633,019 filed Aug. 4, 2000, which is a continuation-in-part of the U.S. patent application Ser. No. 09/390,018 filed Sep. 3, 1999, now U.S. Pat. No. 6,485,667, which is a continuation-in-part of the now abandoned U.S. national stage patent application Ser. No. 09/341,340, filed Jul. 8, 1999 from the international patent application Serial No. PCT/US98/00639, filed Jan. 15, 1998 and which designates the United States, which is a continuation-in-part application of U.S. patent application Ser. No. 08/948,987 filed Oct. 10, 1997, now U.S. Pat. No. 5,916,670, which is a continuation-in-part application of United States patent application Ser. No. 08/784,536 filed Jan. 17, 1997, now U.S. Pat. No. 5,866,242. The disclosures of the prior patent applications are incorporated herein by reference.

### STATEMENT REGARDING FEDERALLY-SPONSORED RESEARCH OR DEVELOPMENT

Not applicable.

### REFERENCE TO MICROFICHE APPENDIX

Not applicable.

### TECHNICAL FIELD

This invention relates to absorbent materials and to a process for making absorbent materials to be used as absorbent cores in articles such as disposable diapers, feminine hygiene products and incontinence devices. More particularly, the present invention relates to improved absorbent materials that are high density, strong, soft materials with superior absorption properties.

### BACKGROUND OF THE INVENTION AND TECHNICAL PROBLEMS POSED BY THE ART

Disposable absorbent articles, such as diapers, feminine hygiene products, adult incontinence devices and the like have found widespread acceptance. To function efficiently, such absorbent articles must quickly absorb body fluids, distribute those fluids within and throughout the absorbent article and be capable of retaining those body fluids with sufficient energy to dry the body surface when placed under loads. In addition, the absorbent article should be sufficiently soft and flexible so as to comfortably conform to body surfaces and provide close fit for lower leakage.

While the design of individual absorbent articles varies depending upon use, there are certain elements or components common to such articles. The absorbent article contains a liquid pervious top sheet or facing layer, which facing layer is designed to be in contact with a body surface. The facing layer is made of a material that allows for the substantially unimpeded transfer of fluid from the body into the core of the article. The facing layer should not absorb fluid per se and, thus, should remain dry. The article further contains a liquid impervious back sheet or backing layer disposed on the outer surface of the article and which layer is designed to prevent the leakage of fluid out of the article.

Disposed between the facing layer and backing layer is an absorbent member referred to in the art as an absorbent core or panel. The function of the absorbent core is to absorb and retain body fluids entering the absorbent article through the facing layer. Because the origin of body fluids is often localized, it is desirable to provide means for distributing fluid throughout the dimensions of the absorbent core to make full use of all the available absorbent material. This is typically accomplished either by providing a distribution member disposed between the facing layer and absorbent core and/or altering the composition of the absorbent core per se.

Fluid can be distributed to different portions of the absorbent core by means of a transition layer, transfer layer, or acquisition layer disposed between the facing layer and core. Because of the proximity of such an acquisition layer to the body surface of the wearer, the acquisition layer should not be formed from material that retains large amounts of fluid. The purpose of the acquisition layer is to facilitate lateral spreading of the fluid, and further to rapidly transfer and distribute the fluid to the absorbent core.

The absorbent core is typically formulated of a cellulosic wood pulp fiber matrix, which is capable of absorbing large quantities of fluid. Absorbent cores can be designed in a variety of ways to enhance fluid absorption and retention properties. By way of example, the fluid retention characteristics of absorbent cores can be greatly enhanced by disposing superabsorbent materials in amongst fibers of the wood pulp. Superabsorbent materials are well known in the art as substantially water-insoluble, absorbent polymeric compositions that are capable of absorbing large amounts of fluid in relation to their weight and forming hydrogels upon such absorption. Absorbent articles containing blends or mixtures of pulp and superabsorbents are known in the art.

The distribution of superabsorbents within an absorbent core can be uniform or non-uniform. By way of example, that portion of an absorbent core proximate to the backing layer (farthest away from the wearer) can be formulated to contain higher levels of superabsorbent than those portions of the core proximate the facing or acquisition layer. By way of further example, that portion of the core closest to the site of fluid entry (e.g., acquisition zone) can be formulated to transport (wick) fluid into surrounding portions of the core (e.g., storage zone).

In addition to blending pulp with superabsorbent material, a variety of other means for improving the characteristics of pulp have been described. For example, pulp boards can be more easily defiberized by using chemical debonding agents (see, e.g., U.S. Pat. No. 3,930,933). In addition, cellulose fibers of wood pulp can be flash-dried prior to incorporation into a composite web absorbent material (see, e.g., U.K. Patent Application GB 2272916A published on Jun. 1, 1994). Still further, the individualized cellulosic fibers of wood pulp can be cross-linked (see, e.g., U.S. Pat. Nos. 4,822,453; 4,888,093; 5,190,563; and 5,252,275). All of these expedients have the disadvantage of requiring the wood pulp manufacturer to perform time-intensive, expensive procedures during the wood pulp preparation steps. Thus, use of these steps results in substantial increases in the cost of wood pulp.

Although all of the above-described treatment steps have been reported to improve the absorption characteristics of pulp for use as absorbent cores, there are certain disadvantages associated with such treatments. By way of example, the manufacturer of the end use absorbent article (e.g. feminine hygiene product or diaper) typically procures wood pulp in the form of a sheet from a wood pulp manufacturer. The end use article manufacturer must then fluff the fibers in the wood pulp sheet so as to detach the individual fibers bound in that pulp sheet. Typically, pulp has a low moisture content, and this causes the individual fibers to be relatively brittle—resulting in fine dust due to fiber breakage during fluffing operations. If the pulp manufacturer performs such fluffing prior to shipment to the absorbent article maker, the transportation costs of the pulp are increased. At least one pulp manufacturer has attempted to solve this problem by producing flash-dried pulp without chemical bonding agents in a narrow range of basis weights and pulp density (see U.S. Pat. No. 5,262,005). However, even with this process, the manufacturer of the absorbent article must still process the pulp after purchase.

There have been numerous attempts by the manufacturers of absorbent materials to produce highly absorbent, strong, soft core materials. U.S. Pat. No. 4,610,678 discloses an air-laid material containing hydrophilic fibers and superabsorbent material, wherein the material is air-laid in a dry state and compacted without the use of any added binding agents. Such material, however, has low integrity and suffers from shake-out or loss of substantial amounts of superabsorbent material. U.S. Pat. No. 5,516,569 discloses that superabsorbent material shake-out can be reduced in air-laid absorbents by adding significant amounts of water to material during the air-laying process. The resultant material, however, is stiff, of low density and has a high water content (greater than about 15 weight percent). U.S. Pat. No. 5,547,541 discloses that high density air-laid materials containing hydrophilic fibers and superabsorbent material can be made by adding densifying agents to the material. The use of such agents, however, increases the production cost of the material.

U.S. Pat. No. 5,562,645 discloses low density absorbent materials (density less than 0.25 g/cc). The use of such low density, bulky materials increases the cost of transportation and handling. U.S. Pat. No. 5,635,239 discloses an absorbent material that contains two complex forming agents that interact when wetted to form a complex. The complex forming agents are polymeric olefins. European Patent Application No. EP 0763364 A2 discloses absorbent material that contains cationic and anionic binders that serve to hold the superabsorbent material within the material. The use of such agents and binders increases the cost of making the absorbent material and poses a potential environmental hazard.

The U.S. Pat. No. 2,955,641 and U.S. Pat. No. 5,693,162 disclose (1) the application of steam to absorbent material to increase the moisture content of the absorbent material, and (2) compressing the absorbent material. The U.S. Pat. No. 5,692,162 also discloses the use of hot calendering rolls (which may be patterned) to form a densified structure, and the use of thermoplastic and thermo-setting resins suitable for thermal bonding.

U.S. Pat. No. 5,919,178 discloses a process for producing an absorbent structure having an intermediate layer containing superabsorbent material sandwiched between two absorbing layers wherein the bottom layer can be a tissue. The patent discloses that when tissue is used as the upper or lower layer, the moisture content of the tissue shall be 20% -70% (by, for example, spraying the tissue with moisture immediately prior to calendaring at a line pressure of 100-200 kg/cm and a temperature of 120° C.-250° C. to compress the web to a density of 0.1 g/cm³ to produce a pulp mat thickness of 1 mm-4 mm).

Nevertheless, there continues to be a need in the art for an improved process for making an absorbent material which satisfies the absorbency, strength and softness requirements needed for use as an absorbent core in disposable absorbent articles and which also provides time and cost savings to both the pulp manufacturer and the manufacturer of the absorbent article.

### BRIEF SUMMARY OF THE INVENTION

According to one aspect of the present invention, a process is provided for making an absorbent material free of added chemical binders and heat set bonding agents. In one embodiment of the material, a web is formed with at least one layer of a mixture of cellulosic fibers and superabsorbent material. The moisture content of the web is increased so as to increase the web density. Preferably, this is affected by adding between about 1% and about 9% moisture by weight based upon the total weight of the web prior to the moisture addition. Then, the web is compacted at an elevated temperature to further increase the web density and to effect hydrogen bonding within the web which is left with substantially no moisture. After the web has cooled, it is preferably maintained at equilibrium in an atmosphere so that the moisture content is between 2% and 8%, and more preferably, between 3% and 6%, by weight based upon the total weight of the web with the moisture.

In one preferred form of the process of the invention, the web moisture content is increased by conveying the web through a region of steam having a temperature above about 100° C., and the web is compacted between calendering rolls wherein at least one of the rolls is heated to a surface temperature in the range of between about 70° C. and about 200° C.

The process of the present invention can be used to make various forms of absorbent material. One form of the absorbent material has a basis weight of from about 180 g/cm² to about 600 g/cm², a density of from about 0.25 g/cc to about 0.45 g/cc. The material is air-laid as a bottom layer of pulp (or pulp and superabsorbent material disposed amongst the pulp), a middle layer of pulp and superabsorbent material disposed in amongst the pulp, and a top layer of pulp. The pulp preferably has a Kappa value of less than about 100. The absorbent material includes from about 40 weight percent to about 90 weight percent cellulosic fibers and from about 10 weight percent to about 60 weight percent superabsorbent material based upon the total weight of the web with the superabsorbent material. Such absorbent material has a water content of less than about 10 weight percent based upon the total weight of the material with the water, and a density of greater than about 0.25 g/cc.

With all forms of the material, it is preferred that at least some of the cellulosic fibers have a relative crystallinity of less than about 65 percent.

In another form, the absorbent material has a basis weight of from about 100 g/m² to about 500 g/m² and a density of from about 0.25 g/cc to about 0.50 g/cc. Such material includes a core of cellulosic fibers obtained from pulp wherein at least some of the pulp fibers have a Kappa value of less than about 100. A carrier layer (e.g., a layer of tissue) may be superimposed on an outer surface of the core. The carrier layer is preferably crepe tissue. At least some of the cellulosic fibers have a relative crystallinity of less than about 65 percent. The core contains from about 40 weight percent to about 100 weight percent cellulosic fibers and from about 0 weight percent to about 60 weight percent superabsorbent material. Preferably, the core contains from about 40 weight percent to about 90 weight percent cellulosic fibers and from about 10 weight percent to about 60 weight percent superabsorbent material.

In another form, the absorbent material has a density of from about 0.25 g/cc to about 0.5 g/cc, and a basis weight of from about 200 g/m² to about 500 g/m². Such material consists essentially of (1) from about 60 weight percent to about 90 weight percent cellulosic fibers at least some of which fibers are obtained from pulp having a Kappa value of less than about 100, wherein at least some of the cellulosic fibers have a relative crystallinity of less than about 60 percent; (2) from about 10 weight percent to about 40 weight percent superabsorbent material; and (3) a layer of tissue comprising from about 3 weight percent to about 20 weight percent of the absorbent material. The tissue is preferably crepe tissue.

Preferably, the material is made using cellulosic fibers having a relative crystallinity of preferably less than about 60 percent. More preferably, the cellulosic fibers have a relative crystallinity of less than about 50 percent and, even more preferably a relative crystallinity of less than about 40 percent. At least some of the cellulosic fibers are obtained from pulp having a Kappa value of less than about 75, 50, 25 or 10. More preferably, the Kappa value is less than 5 or 2.5.

In one form of the absorbent material, at least some of the cellulosic fibers in the material are made by a process that includes the step of treating a liquid suspension of pulp at a temperature of from about 15° C. to about 60° C. with an aqueous alkali metal salt solution having an alkali metal salt concentration of from about 2 weight percent to about 25 weight percent of said solution for a period of time ranging from about 5 minutes to about 60 minutes.

In another form of the absorbent material, at least some of the cellulosic fibers have been flash dried.

In another form of the absorbent material, the cellulosic fibers are not flash dried, but are processed through a hammer mill.

An especially preferred form of absorbent material made by the process of this invention has a density of from about 0.28 g/cc to about 0.40 g/cc, and a basis weight of from about 180 g/m² to about 550 g/m².

According to yet another aspect of the invention, a modification of the process for making an absorbent material includes the steps of

(A) forming a web having a layer of tissue on which is disposed at least one absorbent layer containing cellulosic fibers free of added chemical binders and heat set bonding agents;

(B) spraying water against the web to add between about 2% and about 9% (and preferably between about 1% and about 8%) moisture by weight based upon the weight of the web prior to the moisture addition; and

(C) after step (B), compacting the web at an elevated temperature to further increase the web density, and preferably to also effect either an increase in the strength of one or more of the absorbent layers and/or an increase in the bonding strength of the tissue to the at least one absorbent layer.

The absorbent material produced by the modified process may be characterized as an absorbent material which (I) is made by the process comprising the steps of: (A) forming a web having a first layer of tissue on which is disposed at least one absorbent layer containing cellulosic fibers free of added chemical binders and heat set bonding agents; (B) spraying water against said layer of tissue by adding between about 2% and about 9% (and preferably between 1% and about 8%) moisture by weight based upon the weight of the web prior to the moisture addition; and (C) after step (B), compacting said web at an elevated temperature to further increase the web density and to effect increased bonding of said tissue to said at least one absorbent layer; and (II) has a bond strength between said layer of tissue and the one absorbent layer that exceeds a test delamination force of 3 newtons (as determined pursuant to a test set forth in detail hereinafter).

A second tissue layer may also be bonded to the side of the web opposite from the first layer of tissue.

A preferred form of such an absorbent material may be characterized as a web having a carrier layer on which is disposed at least one absorbent layer containing at least cellulosic fibers free of added chemical binders and heat set bonding agents wherein the web has the following preferred properties:

(1) a density between about 0.25 grams per cubic centimeter and about 0.5 grams per cubic centimeter;

(2) a basis weight between about 150 grams per square meter and about 600 grams per square meter;

(3) a Gurley Stiffness of less than about 1500 milligrams;

(4) a machine direction tensile strength greater than about 9 newtons; and

(5) a bond strength between said at least one absorbent layer and said carrier layer that exceeds a test delamination force of 3 newtons.

Preferred forms of the material have superior absorptive properties. The absorbent material made by the process of this invention can be used to make absorbent articles, such as a diaper, a feminine hygiene product, or an incontinence device.

### BRIEF DESCRIPTION OF THE DRAWINGS

In the drawings, which form a portion of the specification:

FIG. 1 is a simplified, fragmentary, perspective view of a sheet of absorbent material which can be made by the process of the present invention;

FIG. 2 is a greatly enlarged, fragmentary, cross-sectional view taken generally along the plane 2-2 in FIG. 1, and in FIG. 2 the height or thickness of portions of the illustrated structure have been exaggerated for ease of illustration, and it should be understood that FIG. 2 is not necessarily drawn to scale with respect to the thicknesses of the various portions;

FIG. 3 is a diagrammatic view illustrating one form of the process of the present invention;

FIG. 4 is a view similar to FIG. 2, but FIG. 4 illustrates another form of absorbent material;

FIG. 5 is a view similar to FIG. 4, but FIG. 5 illustrates yet another form of an absorbent material;

FIG. 6 is a view similar to FIG. 2, but FIG. 6 illustrates yet another form of the absorbent material;

FIG. 7 is a view similar to FIG. 3, but FIG. 7 illustrates another form of the process of the present invention used to make the absorbent material illustrated in FIG. 6 with an increased bond strength between a tissue layer and the rest of the absorbent core portion;

FIG. 8 is a simplified, front, perspective, fragmentary view of a tensile/compression test machine;

FIG. 9 is a perspective, fragmentary view of the upper platen used in the test machine shown in FIG. 8;

FIG. 10 is a perspective view of a portion of one form of the absorbent material of the present invention to which is attached a length of double-sided tape;

FIG. 11 is a partly diagrammatic, simplified, perspective view of the absorbent material portion of FIG. 10 shown in a hydraulic press employing a circular die to cut a circular test sample;

FIG. 12 is a perspective view of a die cut circular test sample of the absorbent material;

FIG. 13 is a perspective view of the die cut sample mounted to the upper platen before the upper platen is mounted in the test machine;

FIG. 14 is a fragmentary, perspective view of the lower platen of the test machine showing a strip of double-sided tape secured to the lower platen;

FIG. 15 is a fragmentary, side elevational view of the test machine in operation to initially compress the test sample;

FIG. 16 is a fragmentary view similar to FIG. 14; but FIG. 16 shows a later stage during the test wherein the test machine has exerted tension on the test sample and caused the test sample to pull apart; and

FIG. 17 is a typical plot or graph of load versus extension during the test.

### DETAILED DESCRIPTION

The process of the present invention provides an improved absorbent material that is particularly well-suited for use as cores in absorbent articles such as diapers, feminine hygiene products, incontinence devices, and the like. The absorbent material can also be used as an absorbent core in any device used to absorb body exudates (e.g., urine, breast milk, blood, serum). Thus, the absorbent material can be incorporated into breast pads for nursing mothers or used as absorbent material in surgical drapes (e.g., towels) or wound dressings.

The preferred form of the absorbent material includes a blend or mixture of cellulosic fibers and superabsorbent disposed in and amongst fibers of that pulp. The absorbent material has a unique combination of suppleness, strength, and absorbency characteristics that makes it particularly suitable for use in absorbent articles. The absorbent material can be used directly by a manufacturer of the absorbent article without the need for any additional processing by that manufacturer other than cutting or folding the absorbent material to the desired size and shape for the absorbent article.

The process of the present invention can be used to make an absorbent material that is soft, that is thin, and that has relatively high density. Additionally, the material can have enhanced absorption properties, and firmly entraps superabsorbent material in the fiber network without the use of chemicals, binders, adhesives, thermoplastic resins, thermoplastic binder fibers, complex forming materials, or the like. The absorbent material has enough integrity (strength) to be processed on conventional disposable product manufacturing equipment without significant fiber breakage.

According to one aspect of the present invention, the process of the present invention can provide an absorbent material that contains from about 40 weight percent to about 100 weight percent cellulosic fibers based upon the total weight of the absorbent material, from about 0 weight percent to about 60 weight percent superabsorbent material based upon the total weight of the absorbent material, and about 10 weight percent or less water based upon the total weight of the absorbent material.

With reference to the composition of an existing material containing a substance, the phrase “weight percent” as used herein means the weight of the substance divided by the total, combined weight of the substance and material (as determined under ambient conditions) and multiplied by 100. By way of example, absorbent material containing 10 weight percent superabsorbent material means that there are 10 grams of superabsorbent material in a 100 gram specimen containing both the absorbent material and the superabsorbent material.

On the other hand, when reference is made herein to adding a certain weight percent of moisture or water to an existing material, then the weight percent of water being added is based upon the weight of the material prior to the water addition.

Cellulosic fibers that can be used in the process of the present invention are well known in the art and include wood pulp, cotton, flax, and peat moss. Wood pulp is preferred. Pulps can be obtained from mechanical or chemi-mechanical, sulfite, kraft, pulping reject materials, organic solvent pulps, etc. Both softwood and hardwood species are useful. Softwood pulps are preferred. It is not necessary to treat cellulosic fibers with chemical debonding agents, cross-linking agents and the like for use in the absorbent material.

As discussed above, a preferred cellulosic fiber for use in the present material is wood pulp. Wood pulp prepared using a process that reduces the lignin content of the wood is preferred. Preferably, the lignin content of the pulp is less than about 16 percent. More preferably, the lignin content is less than about 10 percent. Even more preferably, the lignin content is less than about 5 percent. Most preferably, the lignin content is less than about 1 percent. As is well known in the art, lignin content is calculated from the Kappa value of the pulp. The Kappa value is determined using a standard, well known test procedure (TAPPI Test 265-cm 85). The Kappa value of a variety of pulps was measured and the lignin content calculated using the TAPPI Test 265-cm 85. Peat moss was found to have a Kappa value of about 104 and a lignin content of about 13.5 percent. CTMP pulp was found to have a Kappa value of about 123 and a lignin content of about 16 percent. Pulp prepared from softwood using either the kraft or sulfite methods had a Kappa value of about 1.1 and a lignin content of about 0.15 percent. When that latter pulp was treated using a cold caustic extraction method, the Kappa value was found to be about 0.97 and the lignin content about 0.12 percent.

For use in the process of the present invention, cellulosic fibers are preferably obtained from wood pulp having a Kappa value of less than about 100. Even more preferably, the Kappa value is less than about 75, 50, 25 or 10. Most preferably, the Kappa value is less than about 2.5.

There are certain other characteristics of wood pulp that make it particularly suitable for use in an absorbent material. Cellulose in most wood pulps has a high relative crystallinity (greater than about 65 percent). In a present material, the use of wood pulp with a relative crystallinity of less than about 65 percent is preferred. More preferably, the relative crystallinity is less than about 50 percent. Most preferably, the relative crystallinity is less than about 40 percent. Also, pulps having an increased fiber curl value are preferred.

Means for treating pulps so as to optimize these characteristics are well known in the art. By way of example, treating wood pulp with liquid ammonia is known to decrease relative crystallinity and to increase the fiber curl value. Flash drying is known to increase the fiber curl value of pulp and to decrease crystallinity. Cold caustic treatment of pulp also increases fiber curl and decreases relative crystallinity. Chemical cross-linking is known to decrease relative crystallinity. It is preferred that the cellulosic fibers used to make the absorbent material by the process of this invention are obtained at least in part using cold caustic treatment or flash drying.

A description of the cold caustic extraction process can be found in commonly owned U.S. patent application Ser. No. 08/370,571, filed on Jan. 18, 1995, which application is a continuation-in-part application of U.S. patent application Ser. No. 08/184,377, filed on Jan. 21, 1994, now abandoned. The disclosures of these two U.S. patent applications are incorporated in their entirety herein by reference thereto.

Briefly, a caustic treatment is typically carried out at a temperature less than about 60° C., but preferably at a temperature less than 50° C., and more preferably at a temperature between about 10° C. and about 40° C. A preferred alkali metal salt solution is a sodium hydroxide solution newly made up or as a solution by-product in a pulp or paper mill operation, e.g., hemicaustic white liquor, oxidized white liquor and the like. Other alkali metals such as ammonium hydroxide and potassium hydroxide and the like can be employed. However, from a cost standpoint, the preferable salt is sodium hydroxide. The concentration of alkali metal salts is typically in a range from about 2 to about 25 weight percent of the solution, and preferably from about 6 to about 18 weight percent. Pulps for high rate, fast absorbing applications are preferably treated with alkali metal salt concentrations from about 10 to about 18 weight percent.

As is well known in the art, flash drying is a method for drying pulp in which pulp is partially dewatered, fiberized, and fed into a stream of hot air which causes the moisture contained in the pulp to be flashed off. Briefly, the pulp, initially at a consistency of 30-45% (containing 55-70% water), is conveyed directly into a fluffer (e.g., a disk refiner) where mechanical action is used to fiberize (break up and separate) and disperse the fibers for the flash drying system. Once discharged from the fluffer device, the fiberized pulp is fed into a flash drying system. The drying system itself is made up of two stages, each of which consists of two drying towers. The fiber is conveyed through the drying towers by high velocities of hot air. The inlet air temperature for the first stage is approximately 240-260° C. while the inlet air temperature for the second stage is approximately 100-120° C. Following each drying stage, the pulp and hot air are then conveyed into a cyclone separator, where the hot air, now containing moisture evaporated from the pulp, is exhausted vertically.

In a typical, small scale system, exhaust temperatures for the first stage, in this case, are approximately 100-120° C., and the exhaust temperatures for the second stage are approximately 90-100° C. At the same time, a material-handling fan draws the pulp fibers through the cyclone cone and on to the next part of the system. Finally, following the second stage cyclone separator, the dried pulp is passed through a cooling stage consisting of a cooling fan which conveys ambient air, and is then passed through a final cooling cyclone separator. The residence time for the entire system, including both drying stages, cyclone separation, and cooling, is approximately 30-60 seconds at the feed rate used (1.5 kg of dry material per minute which is a feed rate typical of a small scale machine). Larger scale. conventional flash drying systems typically have higher feed rates.

A downside to producing flash dried fiber using the type of system described above is the production of localized fiber bundles in the final product. Fiber bundles are formed during the fiberization of the pulp by mechanical action within the fluffer device. The system above uses a disk refiner consisting of two grooved, circular plates at a set gap width, in this case 4 mm. One plate is in a fixed position while the other plate is rotated at high speeds. The pulp is fed into the gap between the two plates and the rotation of the plate results in the separation of fibers along the grooves. Unfortunately, as the pulp is fiberized, some of the individual fibers tend to become entangled with one another, forming small bundles consisting of several individual fibers. As these entangled fibers are flash dried and the moisture is removed, the entanglements tighten and harden to form small localized fiber bundles throughout the flash dried pulp. The presence of large numbers of these localized fiber bundles within the final airlaid products produced using the flash dried pulp can have a deleterious effect on the product physical characteristics and performance. The number of localized fiber bundles can be substantially reduced by using cold caustic extracted pulp.

According to one aspect of the process of the present invention (as described hereinafter), an absorbent material is manufactured to contain a superabsorbent material. Superabsorbent materials are well known in the art. As used herein, the term “superabsorbent material” means a substantially water-insoluble polymeric material capable of absorbing large quantities of fluid in relation to their weight. The superabsorbent material can be in the form of particulate matter, flakes, fibers and the like. Exemplary particulate forms include granules, pulverized particles, spheres, aggregates and agglomerates. Exemplary and preferred superabsorbent materials include salts of crosslinked polyacrylic acid such as sodium polyacrylate. Superabsorbent materials are commercially available (e.g., from Stockhausen GmbH, Krefeld, Germany). A preferred form of the absorbent material contains from about 0 to about 60 weight percent superabsorbent material and, more preferably from about 20 to about 60 weight percent superabsorbent material. Such a preferred form of absorbent material has about 40 to about 100 weight percent cellulosic fibers and, more preferably from about 40 to about 80 weight percent cellulosic fibers.

According to yet another aspect of the present invention, a unique process is provided for manufacturing a web of absorbent material, but the process may be implemented without including a superabsorbent material in all parts of the web or even in any part of the web

Forms of the Absorbent Material

The absorbent material made by the process of the present invention preferably has a moisture content of less than about 10% of the total weight of the absorbent material to discourage bacterial growth. Also, the material is free of added chemical binders and heat set bonding agents.

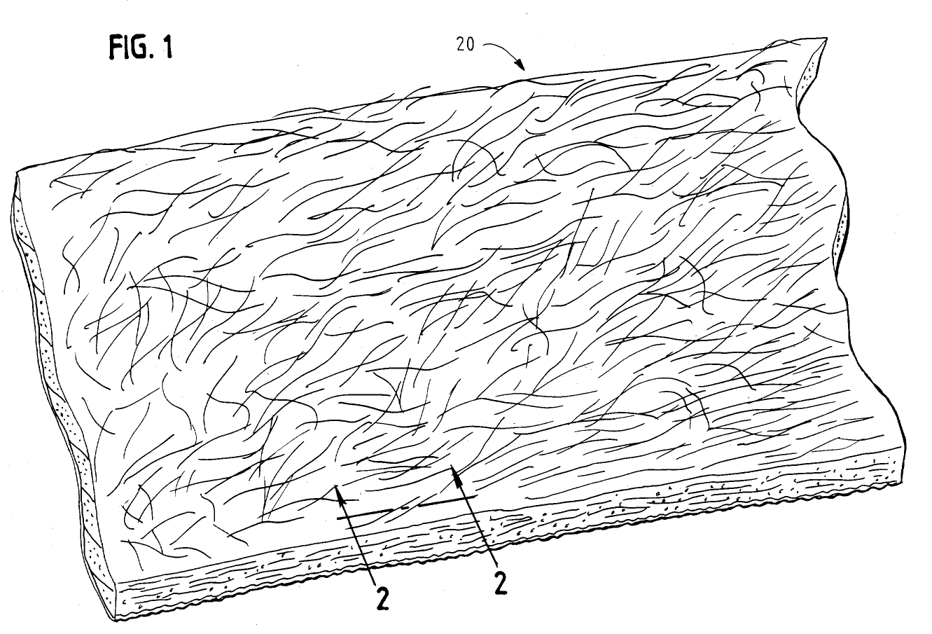

FIG. 1 illustrates one form of an absorbent material that can be made by the process of the present invention. The absorbent material is designated in FIG. 1 generally by the reference number 20. The material 20 is typically made by the process of the present invention in a relatively wide sheet that can be provided in sheet form or in a large roll to a manufacturer of absorbent articles. A typical thickness of the material is between 0.5 mm and 2.5 mm.

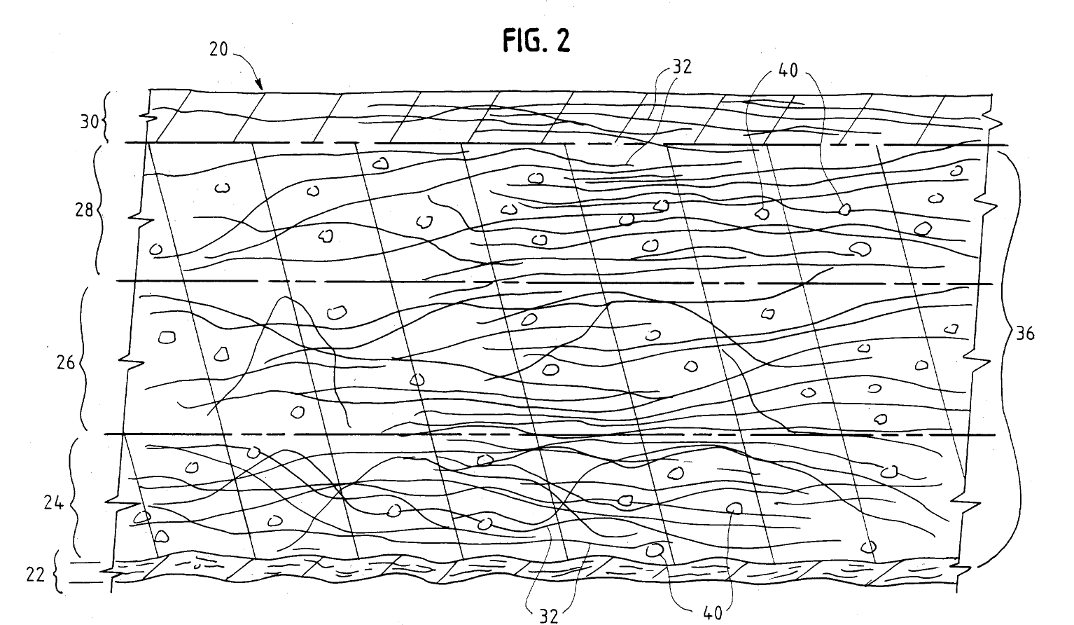

FIG. 2 illustrates a cross section of the material. Regions of various thickness in the material 20 illustrated in FIG. 2 are not necessarily to scale and may in some respects be exaggerated for purposes of clarity and ease of illustration.

The absorbent material 20 illustrated in FIG. 2 includes an optional carrier layer 22. The carrier layer 22 may be, for example, a spunbond, melt blown non-woven consisting of natural or synthetic fibers. Another, and preferred, material that could be used for the carrier layer is tissue.

Suitable tissue materials for use as a carrier layer in absorbent products are well known to those of ordinary skill in the art. Preferably such tissue is made of bleached wood pulp and has an air permeability of about 273-300 CFM (cubic feet minute). The tensile strength of the tissue is such that it retains integrity during formation and calendering of the absorbent material. Suitable MD (machine direction) and CD (cross direction) tensile strengths, expressed in newtons/meter, are about 100-130 and 40-60, respectively. Tissue for use in air-laying absorbent materials are commercially available (e.g., from Cellu Tissue Corporation, 2 Forbes Street, East Hartford, Conn. 06108, U.S.A., and from Duni AB, Sweden). In a preferred embodiment, the tissue is crepe tissue having a sufficient number of crepes per inch to allow a machine direction elongation of between 20 and 35 percent (as determined by the SCAN P44:81 test method).

The absorbent material 20 above the tissue layer 22 typically includes one or more different strata or layers. The thickness of each layer, and the number of layers, may vary. FIG. 2 illustrates a presently contemplated commercial form of the material for a particular application in feminine hygiene-products. The absorbent material 20 illustrated in FIG. 2 includes a bottommost or first layer 24, a second layer 26, a third layer 28, and a fourth layer or top layer 30. In the form of the absorbent material 20 illustrated in FIG. 2, the layers 24, 26, and 28 together define an absorbent core portion 36. The top layer 30, which is optional, is typically characterized as a transition layer, acquisition layer, or transfer layer described in detail hereinafter.

The layers 24, 26, 28, and 30 are referred to as layers or strata because the material forming such layers or strata is initially laid down in the process of the present invention as separate strata or layers one on top of the other as explained in detail hereinafter. However, after completion of the process of making the absorbent material, the layers or strata are part of a unitary or integral structure. Typically, there is little discernable visual difference between the different layers. If one tries to separate the absorbent material into the layers or strata by which it was initially laid down in the manufacturing process, it will be found that the finished absorbent material does not readily pull apart or delaminate into specifically identifiable layers or strata corresponding to the layers or strata laid down during the production process.

Preferably, when a carrier layer, such as tissue layer 22, is used, the tissue layer 22 is lightly embedded into the bottom layer 24 of the absorbent core portion 36, and this can be effected during processing with a knurled roll or linen roll (or other embossed) calendering roll as described in more detail hereinafter. Preferably, if a knurled or linen calendering roll is used, the knurled or linen surface of the roll has a depth greater than 5% of the thickness of the carrier layer (tissue layer).

Typically, an absorbent article manufacturer would add a facing layer, top sheet, or cover stock (not illustrated) over the transition layer 30, and such a facing layer contacts the skin of the person wearing the article. The top, transition layer 30 functions as an acquisition layer to receive liquid (e.g., menses or urine) in the first moments of discharge through the facing layer. This transition layer 30 picks up the liquid from the absorbent article facing layer very quickly and distributes the liquid to the absorbent core 36. The transition layer 30 maintains a distance between the facing layer and the core 36 to inhibit liquid from traveling back from the core 36 to the skin of the wearer of the absorbent article. The transition layer 30 facilitates the lateral spreading of the liquid, especially during second and subsequent discharges of liquid into the absorbent article. In an alternate embodiment, the transition layer 30 may be omitted.

The layers 24, 26, and 28 include pulp fibers 32 which have a typical average length of about 2.40 mm. Preferably, at least some of the pulp fibers 32 are produced by the above-discussed cold caustic extraction process. This includes treating a liquid suspension of pulp containing cellulosic fibers at a temperature of from about 15° C. to about 60° C. with an aqueous alkali metal salt solution having an alkali metal salt concentration from about 2 weight percent to about 25 weight percent of the solution for a period of time ranging from about 5 minutes to about 60 minutes. The treated pulp cellulosic fibers are then either flash-dried or processed through a hammermill.

The absorbent core portion layers 24, 26, and 28 each preferably include a superabsorbent material of the type previously described and which preferably is provided in the form of superabsorbent granules or particles 40. The top, transition layer 30 is free of superabsorbent particles. If the transition layer 30 is omitted, then the top layer of the remaining absorbent core portion layers (e.g., absorbent core portion top layer 28) would preferably be free of superabsorbent particles.

In a preferred form of the absorbent material 20 which is illustrated in FIG. 2 and which has a transition layer 30, each of the absorbent core portion 36 layers 24, 26, and 28 includes superabsorbent particles. If desired, the layers containing pulp and superabsorbent can be laid down as a homogenous blend or as a heterogeneous blend wherein the level of superabsorbent varies with proximity to the bottom (i.e., the bottom carrier layer 22). In modified forms of the absorbent materials, the concentration (weight percent) of superabsorbent material in each layer 24, 26, and 28 can vary, as can the nature or type of the particular superabsorbent material. Also, the superabsorbent material could be limited to only one or some of the layers that constitute the absorbent core pore portion 36.

Preferably, the total basis weight of the pulp and superabsorbent material in the bottom layer 24 is typically between about 50 and about 270 g/m². The total basis weight of the layer 26 is typically between about 50 and about 270 g/m². The total basis weight of the layer 28 is typically between about 20 and about 270 g/m². The total basis weight of the top layer 30 would typically be between about 0 and about 50 g/m².

The preferred thickness of the top, transition layer 30 is in the range of between about 0.20 mm and about 0.50 mm. The preferred thickness of each of the core layers 24, 26, and 28 is in the range of between about 0.2 mm and about 0.9 mm. In some applications, the thickness of each of the core layers may be as low as 0.04 mm or less.

The average density of the absorbent material 20 preferably ranges between 0.25 and 0.5 g/cm³. The moisture content of the absorbent material 20 after equilibration with the ambient atmosphere is preferably less than about 10% (by weight of the total material weight), is more preferably less than about 8%, and preferably lies in the range of between about 3% and about 8%.

Production Process and Apparatus: First Embodiment

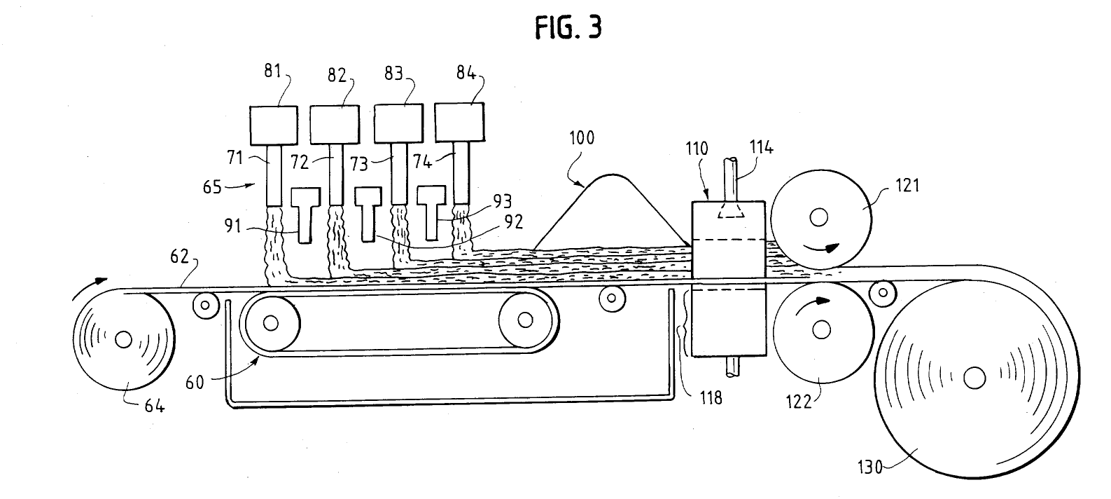

The above-described absorbent materials may be made with the process of the present invention. A first embodiment of the process of the present invention is diagrammatically illustrated in FIG. 3. The illustrated process employs an endless wire, screen, or belt 60 on which the absorbent material components are deposited.

The process permits the optional incorporation of a carrier layer in the absorbent material (e.g., tissue layer 22 in the absorbent material 20 described above with reference to FIG. 2). To this end, a tissue web 62 is unwound from a tissue web roll 64 and directed over the endless screen 60. A series of forming heads 65 is provided over the endless screen 60. In the preferred form of the process, the series includes a first forming head 71, a second forming head 72, a third forming head 73, and a fourth forming head 74. A lesser or greater number of forming heads may be provided depending upon how many layers of material are to be laid down.

Cellulosic fibers, some of which are in the form of the above-described cold caustic extracted pulp fibers, are processed using a conventional hammermill (not illustrated) to individualize the fibers. The individualized fibers are blended with superabsorbent material, granules, or particles in a separate blending system supplying each forming head. The forming head 71 is connected with a blending system 81, the forming head 72 is connected with a blending system 82, and the forming head 73 is connected with a blending system 83. In some systems, the pulp fibers and superabsorbent granules or particles are blended and conveyed pneumatically into the forming heads. In other systems, the pulp fibers and superabsorbent granules or particles are conveyed separately to the forming heads and are blended together in the forming heads. Chemical binding agents and heat set bonding agents are not added during fiber processing or during the blending of the fibers with the superabsorbent material.

The forming head 74 is connected with a blending system 84, and the forming head 74 provides the pulp fibers or other components for the top, transition layer 30. The top layer 30 has no superabsorbent material as previously explained with reference to the absorbent material 20 illustrated in FIG. 2.

The blending and distribution of the materials can be controlled separately for each forming head. For example, in some systems, controlled air circulation and winged agitators in each blending system produce a substantially uniform mixture and distribution (of the pulp and superabsorbent particles for blending systems 81, 82, and 83 and of the pulp fibers alone for blending system 84).

The superabsorbent particles can be either thoroughly and homogeneously blended throughout the absorbent core portion of the structure being produced, or contained only in a specific layer or layers by distributing the superabsorbent particles to selected forming heads.

If desired, the superabsorbent particles can be separately discharged from separate forming heads 91, 92, and 93 as individual layers of 100% superabsorbent. In such an optional configuration, the superabsorbent particle forming head 91 is located between the forming heads 71 and 72, the superabsorbent particle forming head 92 is located between the forming heads 72 and 73, and the superabsorbent particle forming head 93 is located between the forming heads 73 and 74. If the separate superabsorbent particle forming heads 91, 92, 93 are employed, then additional superabsorbent particles could also still be blended in the blending systems 81, 82, and 83. Alternatively, only pulp fibers exclusively could be conveyed to and through the blending systems 81, 82, and 83 and the forming heads 71, 72, and 73, respectively, when superabsorbent material is discharged from the forming heads 91, 92, and 93.

The material from each forming head is deposited by vacuum onto the tissue web or carrier layer 62 (or directly onto the endless screen 60) to form a layered, absorbent web. The layered, absorbent web is conveyed with the help of a conventional vacuum transfer device 100 from the end of the endless screen 60 to a moisture addition apparatus 110. The system could alternatively be designed to eliminate the transfer device 100.

The apparatus 110 typically includes a housing or enclosure and suitable means for supplying moisture and/or controlling the moisture in the enclosure around the layered web. In one contemplated embodiment, the moisture could be supplied in the form of low pressure steam from nozzles on both the top and bottom sides of the layered web, or from just the bottom side. In another contemplated embodiment, the moisture is supplied in the form of low pressure steam through a nozzle 114 above the web. On the side of the web opposite the nozzle 114, the apparatus may include a steam suction chamber 118. Other means may be provided in the device 110 for establishing a desired atmosphere around the layered web wherein the moisture content of the atmosphere is controlled.

In one presently contemplated preferred embodiment of the process of the present invention, the moisture is provided to the device 110 in the form of steam at a pressure of between about 0 psi and about 20 psi and at a temperature of between about 100° C. and about 125° C. The steam is preferably maintained on one side of the web at a temperature of at least about 100° C. The steam flows into and through the layered web and increases the moisture content of the web. According to a preferred form of the first embodiment of the process of the present invention, the moisture content of the web, immediately after being subjected to the steam, is less than about 10 weight percent, preferably less than about 9 weight percent, of the total weight of the web. There is no thermal bonding of the pulp fibers. However, it is also presently believed that the addition of the moisture increases the density of the web and facilitates the establishment of hydrogen bonding of the pulp fibers to each other, as well as of the tissue layer to the pulp fibers, and this increases the strength or integrity of the finished absorbent material.

As the moisturized, layered web exits the moisture addition device 110, it is compressed or compacted between a pair of heated, calendering rolls—upper roll 121 and lower roll 122. This increases the density of the web. In the preferred form where the top layer is free of superabsorbent material, the superabsorbent material in the underlying portion of the web does not contact, and stick to, the heated, upper calendering roll.

The upper roll 121 is typically a steel roll, and the lower roll 122 is typically a flexroll having a hardness of about 85 SH D. Although both rolls could be smooth, in the preferred process, the upper roll 121 has a smooth surface, and the lower roll 122 has a knurled surface. The knurled surface functions to embed the tissue layer 62, or other type of carrier layer, into the bottom of the absorbent material. Preferably, the knurled surface has a depth greater than 5% of the thickness of the carrier layer.

Each roll 121 and 122 is heated to a temperature of between about 70° C. and about 200° C., preferably about 150° C. The weight of the upper roll 121 bears on the layered web. Additional force may be provided with conventional hydraulic actuators (not illustrated) acting on the axle of the roll 121. In one form of the invention, the web is compacted between the rolls 121 and 122 under a load of between about 28 and about 400 newtons per millimeter of transverse web width (160-2284 pounds force per inch of transverse web width). It is presently believed that the heated compaction increases the density of the web and effects the establishment of hydrogen bonding of the pulp fibers to each other, as well as of the tissue layer to the pulp fibers, so as to increase the strength and integrity of the finished absorbent material. This provides a finished product with exceptional resistance to shake-out of superabsorbent material. Upon leaving the calendering rolls, the web contains very little moisture.

The compressed and densified web is wound into a roll 130 using conventional winding equipment. The web moisture content will typically increase as the web reaches equilibrium with the ambient atmosphere, but it is desirable that the moisture content not be too high—preferably be between about 3% and 8% of the total weight of the web.

The processing line is preferably run at a line speed of about 0.5 meters per second to about 5.0 meters per second. The residence time of the web within the moisture addition device 110 is preferably between about 0.1 and about 1.0 seconds.

The presently contemplated preferred form of the first embodiment of the process of the present invention employs calendering rolls 121 and 122 to apply heat and pressure to the web. It will be appreciated, however, that the present invention also contemplates use of other means for compacting and applying heat to the web. For example, a pair of moving, heated platens may be employed in place of the calendering rolls. Alternatively, opposed, heated, endless belt assemblies may be employed to compact and heat the web in place of the calendering rolls. Finally, the instrumentalities for compacting or densifying the web may be separated from the instrumentalities for applying heat to the web. However, it presently appears to be most practical to combine such instrumentalities, as with heated calendering rolls.

Characteristics of the Absorbent Material

A single layer or multilayer absorbent material made by the first embodiment of the process of the present invention is of relatively high density and has a composite density that is preferably greater than about 0.25 g/cc. In preferred embodiments, the absorbent material has a composite density in the range of from about 0.25 g/cc to about 0.50 g/cc. More preferably, the density is from about 0.25 g/cc to about 0.45 g/cc. Most preferably, the density is from about 0.28 g/cc to about 0.40 g/cc.

A high density absorbent material made by the process of the present invention that contains superabsorbent material is surprisingly and unexpectedly supple. The term “supple” is used herein to describe these characteristics of softness, flexibility and bendability. A related characteristic is Gurley stiffness which measures the stiffness of absorbent materials. The greater the value of Gurley stiffness, the more rigid and inflexible the material. The inverse of Gurley stiffness, expressed as inverse grams (g⁻¹), is thus a measure of the softness, bendability and flexibility of absorbent materials. Suppleness is defined and expressed as the inverse of Gurley stiffness and has the units g⁻¹.

The high density absorbent material is strong notwithstanding its suppleness. Pad integrity is a well known characterization of absorbent material strength. The high density absorbent material made by the process of the present invention has good strength (high pad integrity).

An absorbent material can be prepared by the process of the present invention over a wide range of basis weights without adversely affecting its softness or strength. Thus, the absorbent material can have a basis weight in the range of from about 50 g/m² to about 800 g/m² and greater. In a preferred form, the basis weight ranges from about 100 g/m² to about 500 g/m² and, more preferably from about 100 g/m² to about 250 g/m² or from about 300 g/m² to about 500 g/m².

The process of the present invention can be used to make an absorbent material which has superior absorptive properties when compared to existing materials. The absorptive properties of materials can be evaluated in a variety of ways. Of particular relevance to manufacturers of absorbent articles is the ability of the material to absorb large quantities of fluid against a load and to distribute that fluid away from the point of fluid deposition or entry.

Wicking is the ability of an absorbent material to direct fluid away from the point of fluid entry and distribute that fluid throughout the material. An absorbent material made by the process of this invention has good wicking properties.

The unique combination of strength, absorptive capability and suppleness of absorbent material which can be made by the process of the present invention has significant advantages to a manufacturer of absorbent articles. Typically, such a manufacturer purchases pulp, and then processes that pulp on-line in a manufacturing plant as the final article (e.g., diaper, sanitary napkin) is being made. Such processing steps may include defibering of the pulp, adding superabsorbent and the like. In an on-line system, the rapidity with which such steps can be carried out is limited by the slowest of the various steps. An example of a pulp that requires such processing steps (e.g., defibering) is disclosed in U.S. Pat. No. 5,262,005.

The need of the manufacturer to defiberize or otherwise process existing materials on-line means that the overall production process is substantially more complex. Further, the manufacturer must purchase, maintain, and operate the equipment needed to carry out such processing steps. The overall production cost is thus increased.

An absorbent material of the type produced by the process of the present invention can be directly incorporated into a desired absorbent article without the need for such processing steps. The manufacturer of the absorbent article does not have to defiber or otherwise treat the absorbent material made by the process of the present invention in any way other than shaping the absorbent material into the desired shape. In this way, the manufacturer can speed up the assembly process and realize substantial savings in cost and time.

The process of the present invention can be employed to make an absorbent material which also has a good capability to retain superabsorbent material when subjected to mechanical stress.

Forms of Absorbent Material for Use in Feminine Hygiene Products

FIG. 4 illustrates a three-layer form of an absorbent material 300 that can be made by the first embodiment of the process of the present invention and that is particularly well suited for use in feminine hygiene products. Such an absorbent material has a basis weight of from about 100 g/m² to about 250 g/m² and a density between about 0.25 g/cc and 0.5 g/cc. More preferably, the density is from about 0.28 g/cc to about 0.45 g/cc and, most preferably the density is between about 0.28 g/cc and about 0.33 g/cc.

In one variation, the absorbent material 300 for use in a feminine hygiene product is air-laid as three layers or strata: a bottom layer 301 containing pulp (without superabsorbent) with a basis weight of about 25 g/m²; a middle layer 302 with a basis weight of about 150 g/m² and which contains from about 10 g/m² to about 30 g/m² superabsorbent 40C and from about 120 g/m² to about 140 g/m² pulp; and a top layer 303 containing pulp (without superabsorbent) with a basis weight of about 25 g/m². Relative to the total basis weight of the absorbent material 300, the level of superabsorbent 40C ranges from about 5 to about 40 weight percent (g/m² of superabsorbent per g/m² material). Preferably, the level of superabsorbent is from about 7.5 weight percent to about 12.5 weight percent of the absorbent material 300. Most preferably, the absorbent material 300 contains about 30 weight percent of superabsorbent. Thus, the middle layer 302 of the absorbent material 300 preferably contains from about 15 g/m² to about 25 g/m² superabsorbent and from about 125 g/m² to about 135 g/m² pulp and, more preferably about 20 g/m² superabsorbent and about 130 g/m² pulp. The middle layer 302 containing pulp and superabsorbent can be laid down as a homogeneous blend or as a heterogeneous blend wherein the level of superabsorbent varies with proximity to the bottom layer. If desired, superabsorbent could be added to the bottom layer also.

FIG. 5 illustrates another form of an absorbent material 400 wherein the material is air-laid by the process of the present invention as four strata or layers: layer 401, layer 402, layer 403, and layer 404. The layers 402 and 403 may be characterized as two middle layers: a first middle layer 403 adjacent the top layer 404, and a second middle layer 402 adjacent the bottom layer 401. Each of the first and second middle layers independently comprises from about 10 to about 30 g/m² superabsorbent 40D and from about 40 g/m² to about 65 g/m² pulp. When it is desired to keep absorbed fluid away from the top of the feminine hygiene product (i.e., away from the surface of the article in closest proximity to the wearer) the amount of superabsorbent in the first and second middle layers 403 and 402 is adjusted such that there is a higher level of superabsorbent in the second middle layer. The superabsorbent in the first and second middle layers 403 and 403 can be the same or a different superabsorbent. The bottom layer 401 and top layer 404 do not contain any superabsorbent material. However, if desired, superabsorbent could be added to the bottom layer.

Forms of Absorbent Material for Use in Diapers and Incontinent Products

In another variation, the absorbent material made by the first embodiment of the process of the present invention is particularly well suited for use in diapers and incontinence products. Because such articles are expected to absorb and retain larger quantities of less viscous fluid than a feminine hygiene article, such an article employs absorbent material which is heavier and, thus, has a preferred basis weight of from about 300 g/m² to about 500 g/m². The overall composite density of that material is between about 0.3 g/cc and 0.5 g/cc. More preferably, the overall composite density is from about 0.35 g/cc to about 0.45 g/cc and, most preferably about 0.38 g/cc.

In a manner similar to that described above for a feminine hygiene product, a material suitable for use in diapers can be air-laid as one layer or multiple strata such as two, three, four, or more strata. When three strata are used (FIG. 4), the bottom layer 301 has a basis weight of about 50 g/m²; the middle layer 302 has a basis weight of about 300 g/m² and contains from about 40 g/m² to about 200 g/m² superabsorbent 40C and from about 100 g/m² to about 260 g/m² pulp; and the top layer 303 has a basis weight of about 50 g/m². Preferably, the middle layer contains from about 70 g/m² to about 170 g/m² superabsorbent and from about 130 g/m² to about 230 g/m² pulp. Even more preferably, the middle layer 302 contains about 80 g/m² superabsorbent and about 220 g/m² pulp or about 160 g/m² superabsorbent and about 140 g/m² pulp. The middle layer containing pulp and superabsorbent can be laid down as a homogeneous blend or as a heterogeneous blend wherein the level of superabsorbent varies with proximity to the bottom layer. If desired, superabsorbent could be added to the bottom layer.

In a four strata variation (FIG. 5) used for diapers and adult incontinence products, the absorbent material 400 has two middle layers 403 and 403 which each independently contains from about 20 g/m² to about 100 g/m² superabsorbent 40D and from about 50 g/m² to about 130 g/m² pulp. In a preferred embodiment, the second (lower) middle layer 402 has a higher level of superabsorbent 40D than the first (upper) middle layer 403. In this way, the formed absorbent material 400 has a tendency to keep absorbed fluid away from the body surface of the wearer of the article. The superabsorbent 40D in the first and second middle layers 403 and 402 can be the same or a different material. If desired, superabsorbent could be added to the bottom layer.

An absorbent material made by the process of the present invention can be incorporated into an absorbent article as a single-ply or multiple-ply structure. Means for forming such structures using folding are well known in the art. By way of example, a person skilled in art can “C-fold”, “G-fold,” or “Z-fold” the absorbent material prior to incorporating it into an absorbent article.

Modified Form of the Absorbent Material

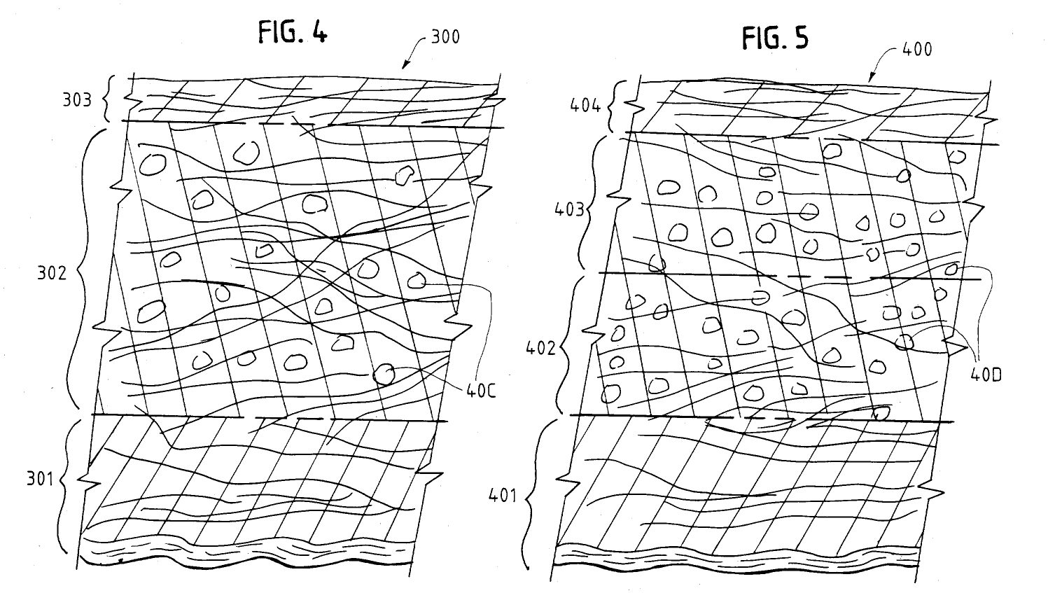

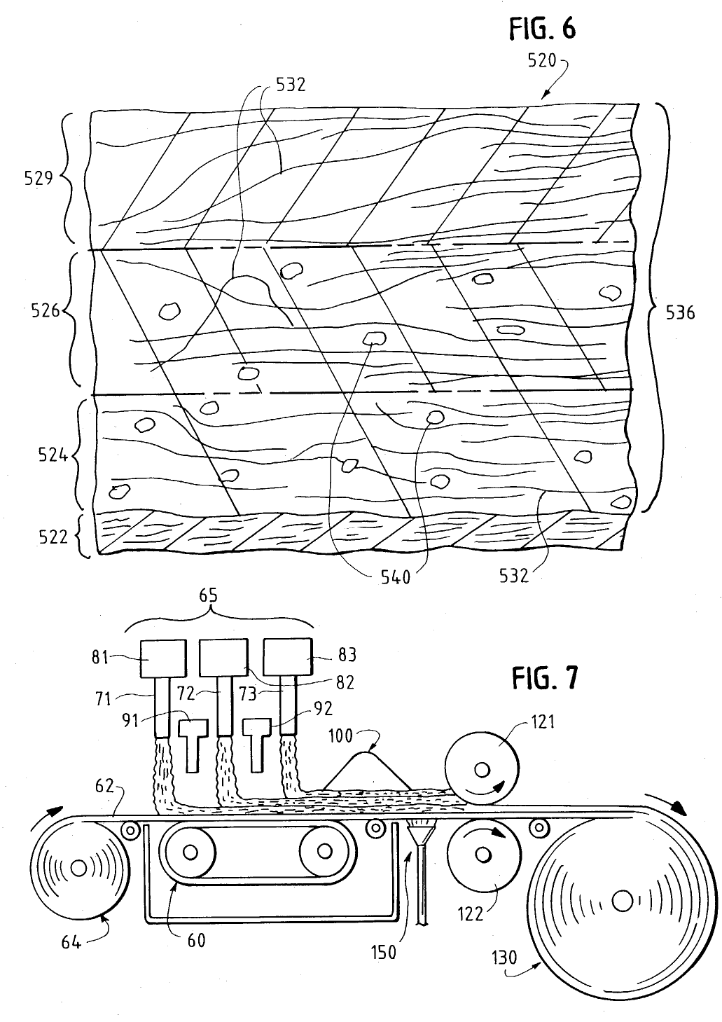

FIG. 6 illustrates another form of an absorbent material 520 which can be made by another embodiment of the process of the present invention. The absorbent material 520 includes a tissue layer or carrier layer 522, a first layer 524, a second layer 526, and a third layer or top layer 529. In the form of the absorbent material 520 illustrated in FIG. 6, the layers 524, 526, and 529 together define an absorbent core portion 536 having a tissue carrier or tissue layer 522. In this particular embodiment, there is no transition layer or acquisition layer. However, that is an optional layer that may be provided, and such a layer may have the structure and function which is identical with the structure and function of the transition layer 30 described above with reference to the first embodiment illustrated in FIG. 2.

As with the layers of the first embodiment illustrated in FIG. 2 and described above, the layers 525, 526, and 529 of the embodiment shown in FIG. 6 are referred to as layers or strata because the material forming such layers or strata is initially laid down in a form of the process of the present invention as separate strata or layers one on top of the other. One form of an apparatus or processing line for producing the absorbent material 520 illustrated in FIG. 6 is described hereinafter with reference to FIG. 7.

After completion of the process of making the absorbent material 520, the layers or strata are part of a unitary or integral structure. Typically, there is little discernable visual difference between the different layers. If one tries to separate the absorbent material into the layers or strata by which it was initially laid down in the manufacturing process, it will be found that the finished absorbent material does not readily pull apart or delaminate into specifically identifiable layers or strata corresponding to the layers or strata laid down in the production process.

In the absorbent material 520, it is especially desirable that the tissue layer 522 be effectively and strongly bonded to the absorbent core portion bottom layer 524 without adversely or deleteriously affecting the physical properties of the material 520, such as pad integrity, softness, bendability, flexibility, suppleness, and tensile strength.

The tissue layer 522 may have the same composition as the tissue layer 22 in the first embodiment of the absorbent material 20 described above with reference to FIG. 2. For the specific embodiment of the absorbent material 520 illustrated in FIG. 6, the tissue layer 522 may be of a conventional type made from 100% southern softwood bleached pulp.

The absorbent core portion layers 524, 526, and 529 each include pulp fibers 532 in a form as described above for the pulp fibers 32 of the first embodiment of the absorbent material 20 illustrated in FIG. 2. The absorbent material 520 preferably includes superabsorbent particles or granules 540 in the first layer 524 and in the second layer 526 of the absorbent core portion 536. Such superabsorbent particles 540 are dispersed throughout the two layers 524 and 526 in substantially the same way that the superabsorbent particles 40 are dispersed in the layers 24 and 26 of the first embodiment of the absorbent material 20 described above with reference to FIG. 2.

In the preferred embodiment of the absorbent material 520 illustrated in FIG. 6, the third layer or top layer 529 does not include any superabsorbent particles 540. Although superabsorbent particles could be provided in the top layer 529, it is preferred in many applications that the top layer 529 be free of superabsorbent particles. The concentration (weight percent) of superabsorbent material 540 in each layer vary, as can the nature or type of the particular superabsorbent material.

Production Process and Apparatus: Modification

The absorbent material 520 illustrated in FIG. 6 can be made with the embodiment of the apparatus and process illustrated in FIG. 3 and described above with reference to making the absorbent material 20 illustrated in FIG. 2 as well as with a modified form of that apparatus and process. Such a modified form of the apparatus and process for making the absorbent material 520 is illustrated in FIG. 6 is shown in FIG. 7.

The process accommodates the incorporation of a carrier layer in the absorbent material (e.g., tissue layer 522 in the absorbent material 520 described above with reference to FIG. 6). To this end, a tissue web 62 is unwound from a tissue web roll 64 and directed over the endless screen 60 as shown in FIG. 7. A series of forming heads 65 is provided over the endless screen 60 to deposit cellulosic fibers. In the preferred form of the process, the series includes a first forming head 71, a second forming head 72, and a third forming head 73. A lesser or greater number of forming heads may be provided depending upon how many layers of material are to be laid down.

The cellulosic fibers preferably include fibers from a blend of (1) 100% southern pine Kraft processed pulp, and (2) the above-described cold caustic treated Kraft processed pulp (e.g., cold caustic extracted pulp fibers). The fibers are processed using a conventional hammermill (not illustrated) to individualize the fibers. The individualized fibers are preferably blended with superabsorbent material, granules, or particles in separate blending systems supplying the forming heads 71 and 72. The forming head 71 is connected with a blending system 81, and the forming head 72 is connected with a blending system 82. In some systems, the pulp fibers and superabsorbent granules or particles are blended and conveyed pneumatically into the forming heads 71 and 72. The forming head 73 may have a blending system 83, but the blending system 83 is not operated to blend superabsorbent particles if the top layer 529 of the absorbent material 520 is to be made without superabsorbent. In other systems, the pulp fibers and superabsorbent granules or particles are conveyed separately to the forming heads and are blended together in the forming heads. Chemical binding agents and heat set bonding agents are preferably not added during fiber processing or during the blending of the fibers with the superabsorbent material.

The blending and distribution of the materials can be controlled separately for each forming head. For example, in some systems, controlled air circulation and winged agitators in each blending system produce a substantially uniform mixture and distribution (of the pulp fibers and superabsorbent particles for blending systems 81 and 82, and of the pulp fibers alone for blending system 83).

The superabsorbent particles can be either thoroughly and homogeneously blended throughout the absorbent core portion of the structure being produced, or contained only in a specific layer or layers by distributing the superabsorbent particles to selected forming heads.

If desired, the superabsorbent particles can be separately discharged from separate forming heads 91 and 92 as individual layers of 100% superabsorbent. In such an optional configuration, the superabsorbent particle forming head 91 is located between the forming heads 71 and 72, and the superabsorbent particle forming head 92 is located between the forming heads 72 and 73. If the separate superabsorbent particle forming heads 91 and 92 are employed, then additional superabsorbent particles could also still be blended in the blending systems 81 and 82. Alternatively, only pulp fibers exclusively could be conveyed to and through the blending systems 81 and 82 and the forming heads 71 and 72, respectively, when superabsorbent material is discharged from the forming heads 91 and 92.

The material from each forming head is deposited with vacuum assist onto the tissue web or carrier layer 62 to form a layered, absorbent web. The layered, absorbent web can be, but need not be, conveyed with the help of a conventional vacuum transfer device 100 from the end of the endless screen 60 to a moisture addition spray apparatus 150. The steam addition enclosure 110 employed in the first embodiment of the process illustrated in FIG. 3 is eliminated from the second embodiment of the process shown in FIG. 7. The system could alternatively be designed to also eliminate the transfer device 100.

The spray apparatus 150 includes six water spray nozzles across the width of the web. The number of nozzles may be varied depending on the size or width of the processing line. Each nozzle is oriented to direct a conical pattern water spray against the underside of the tissue layer web 62. Each nozzle is a Variable Spray Autojet Automatic Air Atomizing Nozzle Model No. 1/8 VAA-SS+SUV67-SS sold by Spraying Systems Co., North Avenue at Schmele Road, P.O. Box 7900, Wheaton, Ill. 60189, U.S.A. Other types of nozzles could be used.

The spray nozzles are located about 10 inches below the tissue layer web and are spaced apart across the width of the web on 30 cm centers (i.e., the distance between adjacent nozzles is 30 cm). The water pressure is adjusted to provide the desired amount of liquid sprayed against the web. In a presently preferred form of the process, the water pressure is maintained at about 15 psig to achieve a 3% moisture addition by weight (based upon the weight of the absorbent material web prior to addition of the moisture). The water temperature is maintained at about 15° C. The temperature of the water could be higher or lower if desired. The type, arrangement, operation, and number of the nozzles may be varied depending upon the type of tissue, the composition and construction of the absorbent core portion 536, size of the processing line, the line speed, the nature of the downstream processing, and the like.

In the presently contemplated preferred embodiment of the process illustrated in FIG. 7, the moisture content of the web is increased to effect a bonding of the tissue layer web 62 (finished product layer 522 in FIG. 6) to the bottom layer of the absorbent core portion without thermal bonding of the pulp fibers.

After the moisturized, layered web passes beyond the moisture addition spray apparatus 150, it is compressed or compacted between a pair of heated, calendering rolls—upper roll 121 and lower roll 122. This increases the density of the web. In the preferred form where the top layer is free of superabsorbent material, the superabsorbent material in the underlying portion of the web does not contact, and stick to, the heated, upper calendering roll.

The upper roll 121 is typically a steel roll, and the lower roll 122 is typically a flexroll having a hardness of about 85 SH D. Although both rolls could be smooth, in the preferred process, the upper roll 121 has a smooth surface, and the surface of the lower roll 122 is not smooth. The lower roll could have a knurled surface. Preferably, the lower roll 122 is characterized as a “linen roll” having a non-smooth surface which has the three-dimensional configuration or impression of a linen-like fabric. The non-smooth surface functions to embed the tissue layer web 62, or other type of carrier layer, into the bottom of the absorbent material. Preferably, the indentations of the linen roll surface have a depth greater than 5% of the thickness of the carrier layer 522.

One form of a linen roll that has been used to make samples of the invention described hereinafter in the section entitled “EXAMPLES” is a linen roll designated by the Design Number 204RE87 and sold by Saveressig GmbH & Co., Gutenbergstrasse 1-3, D-48691 Vreden, Germany. The roll is made from the metal alloy 42 CrMo and is not nitrated. The roll diameter is 500 mm, and the roll bale width is 1,950 mm. The gravour width is 1,850 mm, and the gravour depth is 300 μm.

Each roll 121 and 122 is preferably heated to a temperature of between about 70° C. and about 200° C., more preferably to about 150° C. The weight of the upper roll 121 bears on the layered web. Additional force may be provided with conventional hydraulic actuators (not illustrated) acting on the axle of the roll 121. In one form of the invention process, the web is compacted between the rolls 121 and 122 under a preferred load range of between about 160 and about 2284 pounds force per linear inch of transverse web width, preferably about 515 pounds force per linear inch.

It is presently believed that the heated compaction increases the density of the web and effects the establishment of hydrogen bonding of the pulp fibers to each other within the layers of the absorbent core portion 536 (FIG. 6), as well as of the tissue layer 522 to the pulp fibers in the bottom layer 524 of the absorbent core portion 536. The compaction also increases physical entanglement of the fibers. These factors increase the delamination strength of the bond between the absorbent core portion 536 and the tissue layer 522. This also increases the strength and integrity of the finished absorbent material 520. This prevents the tissue layer 522 from delaminating from the bottom layer 524 of the absorbent core portion 536 during use of the material, such as during manufacture of an absorbent product employing the absorbent material 520. This also provides a finished product with exceptional resistance to shake-out of superabsorbent material. Surprisingly, however, there is no significant adverse effect on softness, suppleness, and absorbency.

One preferred range of the processing line speed is between about 30 and about 300 meters per minute, and the preferred speed is 90 meters per minute.

The compressed and densified web is wound into a roll 130 using conventional winding equipment.

A single layer or multilayer absorbent material 520 made by the embodiment of the process of the present invention illustrated in FIG. 7 is of relatively high density and has a composite density that is preferably greater than about 0.25 g/cc. In preferred embodiments, the absorbent material 520 has a composite density in the range of from about 0.25 g/cc to about 0.50 g/cc. More preferably, the density is from about 0.28 g/cc to about 0.45 g/cc.

An absorbent material can be prepared by the embodiment of the process of the present invention shown in FIG. 7 over a wide range of basis weights without adversely affecting its softness or strength. Thus, the absorbent material can have a basis weight in the range of from about 50 g/m² to about 800 g/m² and greater. In a preferred form, the basis weight ranges from about 100 g/m² to about 500 g/m².

The embodiment of the process of the present invention shown in FIG. 7 can be used to make an absorbent material which has superior absorptive and wicking properties when compared to existing materials.

The form of the absorbent material which is made by the embodiment of the process illustrated in FIG. 7 has a tissue layer that is bonded to the absorbent core portion with relatively high strength. The unique combination of a high strength tissue bond and overall material strength, absorptive capability, good wicking properties, and suppleness of the absorbent material, which can be made by the embodiment of the process shown in FIG. 7, has significant advantages to a converter or manufacturer of absorbent articles. The absorbent material can be readily handled and processed as the final article (e.g., diaper or sanitary napkin) is being made, and the absorbent material will withstand such handling without the tissue pulling away and without the material otherwise suffering a significant loss of the integrity. As a result, the manufacturer can process the absorbent material with better runnability and decreased breakage.

Samples of the absorbent material 520 illustrated in FIG. 6 were tested to evaluate the material integrity strength vis-a-vis the strength of the bond between the tissue layer 522 and the bottom layer 524 of the absorbent core portion 536. Some of the samples were made by the process generally illustrated in FIG. 7. Other samples were also made by the process shown in FIG. 7 except that water was not sprayed onto the tissue layer, and the calendering conditions were varied. Still other samples were made by removing tissue from a previously produced absorbent material, applying a layer of new tissue, and then in a laboratory adding water and heating the sample in a flat press.

As explained hereinafter in detail, the samples were tested for resistance to rupture or delamination when subjected to a tensile force applied perpendicularly to the plane of the sample. As set forth hereinafter in detail, the test results were recorded and compared. Analysis of the results shows that addition of water improves bonding between the tissue layer and the bottom layer of the absorbent tissue portion. Also, the bonding is improved somewhat by use of particular calendering conditions.

Delamination Test Procedure for Determining the Delamination Strength of an Absorbent Core Portion with a Tissue Layer

The delamination or rupture tensile strength of the samples was determined according to a test procedure set forth below. The test is used to estimate the extent of bonding that exists between the carrier tissue layer and the rest of the absorbent core portion (pulp layers) of a sample of absorbent material. If, when a tensile force is applied to the sample perpendicularly to the plane of the sample, the sample ruptures or pulls apart at the interface between the tissue and absorbent core portion, then the bond strength corresponds directly to that maximum applied tensile force. However, if the sample ruptures within the core instead of at the tissue/core interface, then the bond strength at the tissue core interface is at least as great as the core rupture strength.

An Instron brand tensile/compression test machine is used to apply a tensile force to a 2 inch diameter circular sample of a laminate of a tissue layer and an absorbent core portion, and the force is applied perpendicularly to the plane of the sample. Tension forces are applied on one side of the sample at the pulp surface and on the other side of the sample at the oppositely facing carrier tissue surface until failure occurs. The force is transmitted from the test apparatus to the oppositely facing surfaces of the sample secured with double-sided tape to metal platens of the machine as explained in detail hereinafter.

This delamination test quantifies the tension force necessary to rupture the sample within the absorbent core portion—or at the interface of the carrier tissue and absorbent core portion if that interface bond strength is less than the strength of the absorbent core portion per se. The extent of bonding, or strength of bonding, between the tissue layer and the pulp of the absorbent core portion is dependent upon many variables. A minimum level of bonding or adhesion is desired to maintain product integrity. This is especially true for material that is subsequently processed at converting machines wherein the material is incorporated in absorbent article products such as diapers or feminine hygiene articles.

The following items are used in the test:

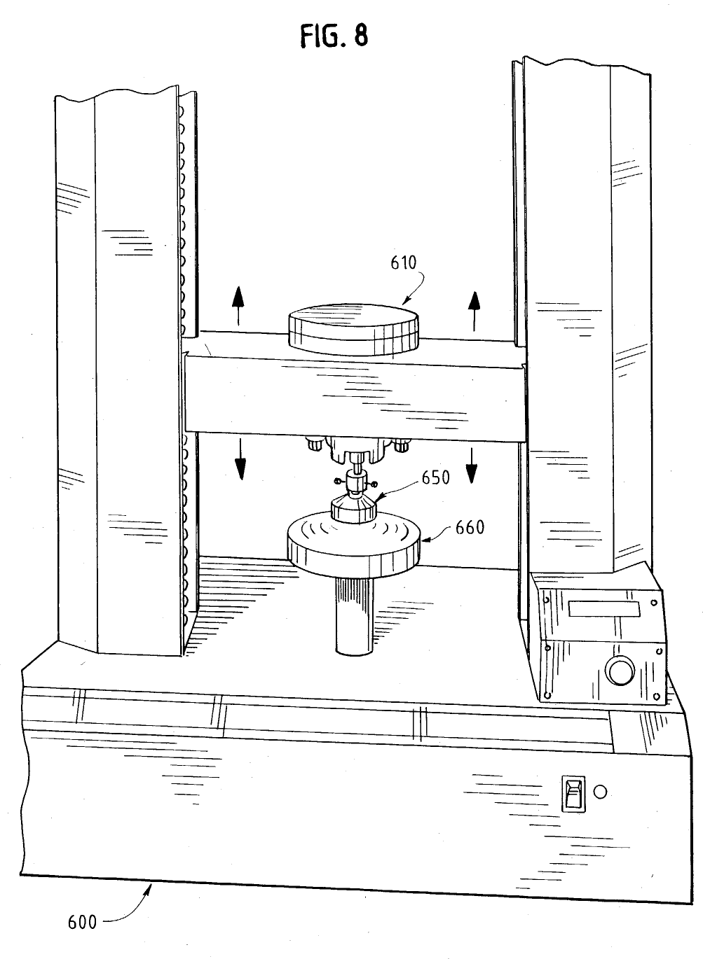

a) Model 554201 Instron brand tensile/compression test machine (reference number 600 in FIG. 8) and a dedicated computer loaded with the Merlin brand software package as provided by Instron;

b) 50 newton tensile/compression load cell (reference number 610 in FIG. 8);

c) Instron brand pivot joint for the upper fixture platen;

d) Model ATOM—SE 15 Hudson Machinery Worldwide hydraulic press (reference number 620 in FIG. 11);

e) 2 inch diameter circular die (reference number 630 in FIG. 11);

f) 3M Scotch brand double-sided tape (#41010DSLD01AC502D);

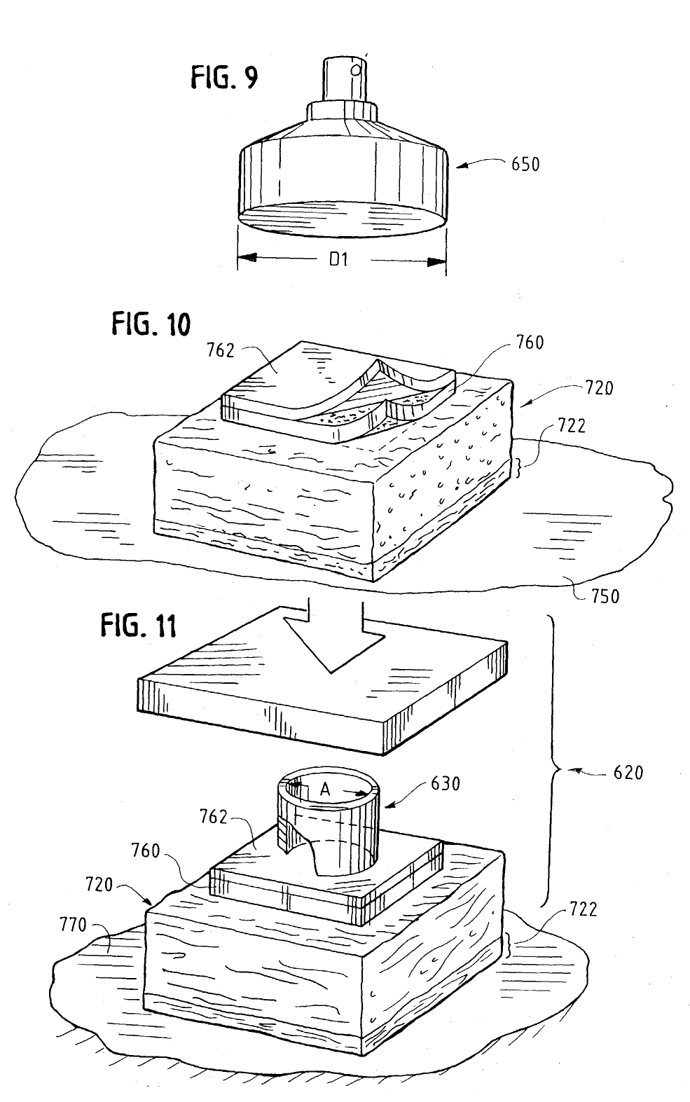

g) circular brass upper platen (reference number 650 in FIGS. 8 and 9) having a platen face with a diameter D1 of 1.978 inches; and

h) lower platen (reference number 660 in FIG. 8) with a diameter greater than 2 inches.

First, the material from which the samples are to be taken is conditioned in a controlled environment at a temperature of 23±1° C. and a relative humidity of 50%±2% for a minimum of 2 hours. The density of the sample material is determined by calculating the volume of a specimen as the arithmetic product of the measured thickness, length, and width of the specimen, and then dividing the measured weight by the volume of the specimen.

A strip of the sample material is prepared so that it is at least 18 inches in length and greater than 2 inches in width. Alternatively, enough smaller sections of the sample material 720 (FIG. 10) are obtained so that seven (7) circular samples, each with a 2 inch diameter, can be created from it.

Next, the sample material 720 is placed with the tissue layer 722 side down on a support surface 750 (FIG. 10). The release paper is removed from only one side of a length of Scotch brand 3M double-sided tape 760 (FIG. 10). The exposed adhesive side of the tape 760 is applied to the upwardly facing, exposed pulp side of the sample material 720 (FIG. 10). The top surface release paper 762 (FIG. 10) is left in place on the tape 760 at this time.

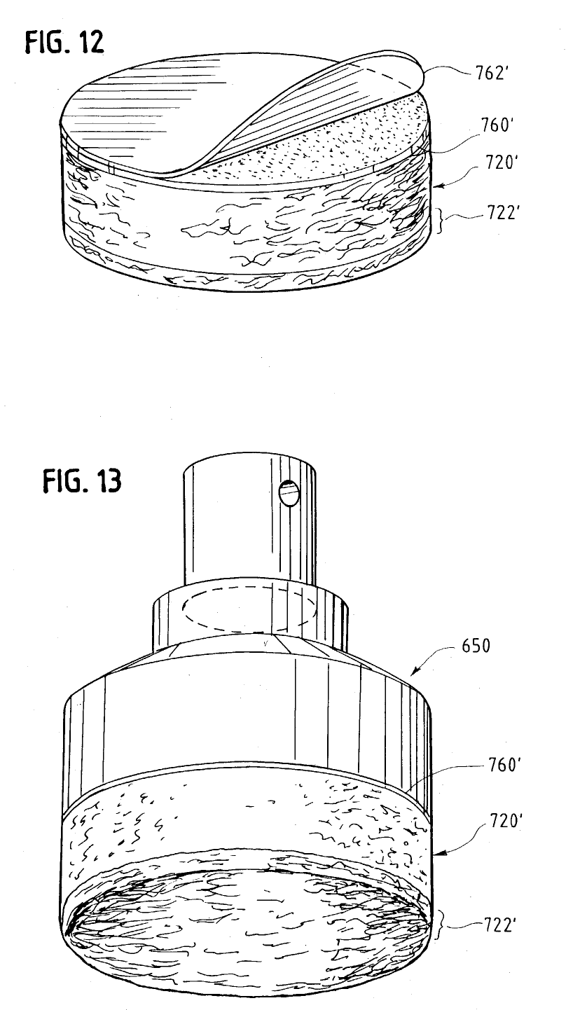

Then, as shown in FIG. 11, the sample material 720 with the attached tape 760 is placed on the bed 770 of the hydraulic press so that the carrier layer (tissue layer) side is down, and the tape release paper side is facing upwardly. A circular sample 720′ (FIG. 12) is die cut using the 2 inch diameter circular die 630 with the hydraulic press 620. The die cut sample 720′ has die cut tissue 722′ on the bottom and has a die cut piece of tape 760′ and release paper 762′ still attached to the top so that the sample 720′ can be mounted to the test machine later, as explained below, following set up of the test machine.

For typical samples having a rupture or delamination tensile strength between 2 newtons and 11 newtons, a 50 newton tensile/compression load cell 610 (FIG. 8) is installed on the Instron brand test machine crosshead to provide adequate sensitivity and at the same time have a high enough rating to withstand the initial compression loading when the upper platen 650 is initially moved down to first force the sample 720′ against the lower platen 660 between tapes to cause the sample 720′ to adhere to the platens as described in detail below.

The circular, metal, lower platen 660 (FIG. 8), having a diameter greater than 2 inches (e.g., 8 inches), is used as the Instron brand machine lower fixture.

The computer is started, and Merlin brand software is launched. The vertical delamination test, which is listed as a compression method, is selected to activate the Instron brand machine. The following “Pre-Cycle” parameters are set:

Crosshead Speed: 75 mm/minute; and

Maximum Criteria: 35 newtons load (a compression load sufficient to ensure that there is a strong taped securement of the sample 720′ to the upper and lower platens as described in detail below).

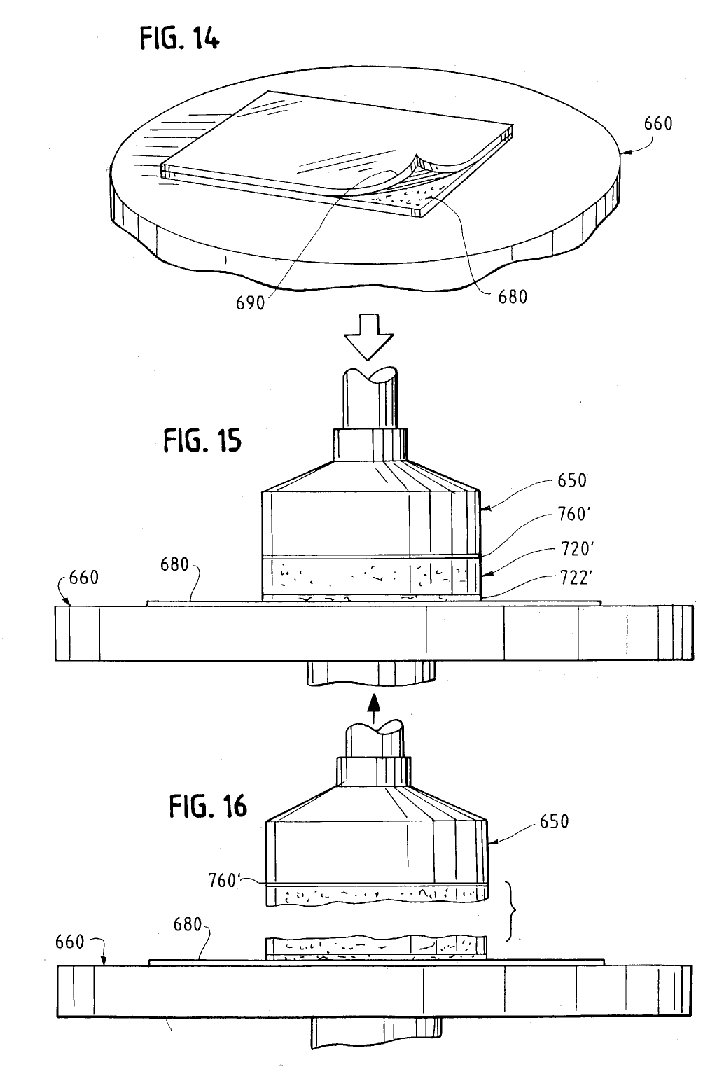

A second strip of the Scotch brand 3M double-sided tape 680 (FIG. 14) is placed on the lower platen 660. Specifically, the release paper is removed from the bottom side of the strip of tape 680, and the sticky bottom side of the tape 680 is adhered to the lower platen 660 (FIG. 14) in a location so that when the upper platen 650 is later installed, the entire surface of the upper platen will be in vertical registry with the second strip of tape 680 on the lower platen 660. Then, the release paper 690 is removed from the upwardly facing top side of the second strip of tape 680 on the lower platen 660.

The previously prepared die cut sample 720′ is next attached to the upper platen 650 (FIG. 13). This is effected by removing the top side release paper 762′ from the tape 760′ on the sample 720′ so as to expose the adhesive side of tape 760′, and then pressing the sample with the attached tape to the surface of the 2 inch diameter, upper, circular, brass platen 650.

The upper, circular, brass platen 650 with the attached sample 720′ is then mounted with the retaining pin in the self-aligning grip coupling (Instron catalog No. 2301-115) in the upper fixture of the Instron brand machine 600.

The Instron brand machine 600 is then adjusted to locate the upper, circular, brass platen 650 so that it is 2.5 inches from the lower, circular platen 660.

The Instron brand machine gauge length is then set.

The setup is checked to make sure that any random sample movement is minimal.

The load of the Instron brand machine is balanced by pressing the “Balance” switch.

The test is started by pressing the “Start” switch.

During the test procedure, the upper platen 650 will initially move downwardly toward the lower platen 660 until the sample 720′ is compressed with a force of 35 newtons (FIG. 15) to create a bonding of the upper platen 650, double-sided tape 760′, and specimen 720′, and to create a bonding of the lower platen 660, double-sided tape 680, and specimen 720′. After the 35 newton compressive force is reached, the movement direction of the upper platen 650 will automatically reverse, and the upper platen 650 will move upwardly away from the lower platen 660 at a rate of 75 mm/minute, and the sample will rupture or delaminate (FIG. 16).

During the movement of the platen 650 away from the platen 660, the sample 720′ will be stressed in the tensile mode. The maximum magnitude of the tensile force during the tensile mode will correspond to the extent of bonding between the ruptured or delaminated portions of the sample 720′ (FIG. 16). This can indicate either the strength of the bond between the tissue (carrier layer) and the core (if the tissue delaminates from the core), or the strength of the core per se where the core per se ruptures the (if the strength of the bond between the carrier layer and core is greater than the strength of the core per se.

After the sample 720′ has ruptured or delaminated, the upper platen 650 is removed from the Instron brand machine 600, and the sample pieces are removed from the upper and lower platens.

A new sample can be prepared and mounted to the upper platen as described above, and the upper platen can then be re-installed on the Instron brand machine and returned to the gauge length by pressing the “Return” switch.

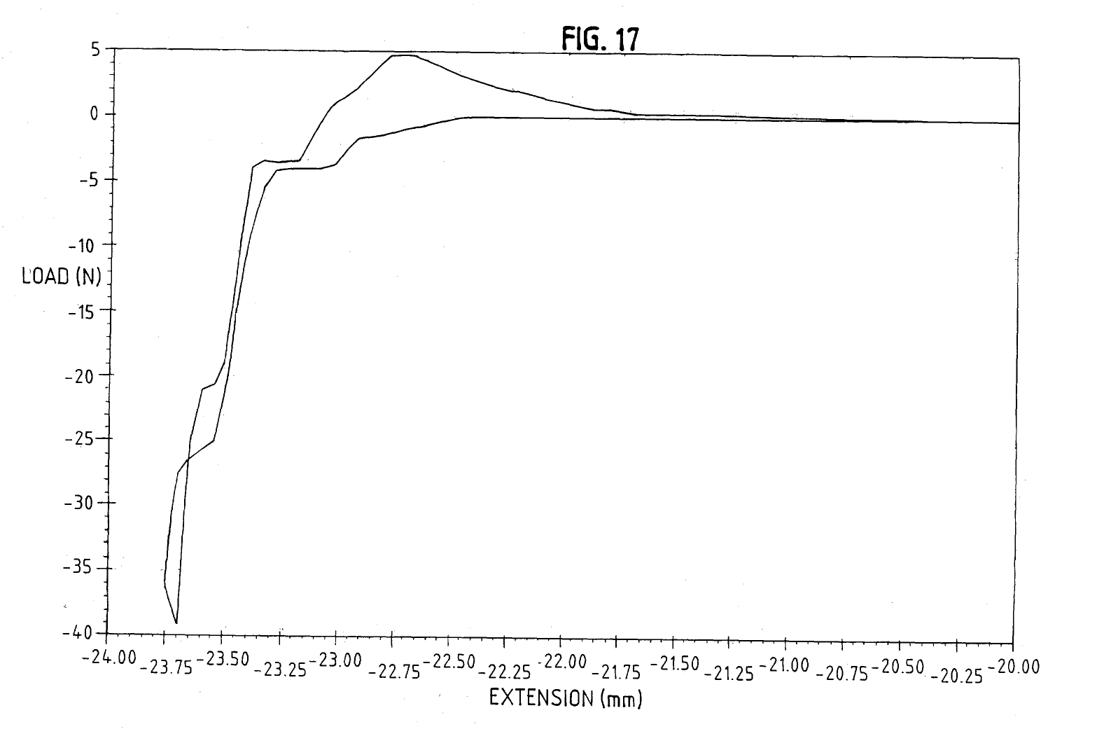

Seven samples are tested. The rupture or delamination force (which is the maximum positive value in the force curve generated by the graph plotting software) for each of the seven samples is recorded, and the average value is calculated. A typical force curve is shown in FIG. 17.

Other Tests and Measurements

Other physical characteristics of the material from which the rupture or delamination strength test samples were taken are determined by additional testing.

Basis Weight Determination

The basis weight of the absorbent material from which the delamination strength test samples are taken is determined from a specimen of the material by first weighing the specimen. The length and width of the specimen is the measured. The length and width are multiplied to calculate the area. The weight is then divided by the area, and the quotient is the basis weight.

Density Determination

The density of the absorbent material from which the delamination strength test samples are taken is determined from a specimen of the material by first weighing the specimen. The length, width, and thickness are measured and multiplied together to calculate the volume. The specimen weight is then divided by the volume to calculate the density.

Gurley Stiffness Determination

The “Gurley Stiffness” of the absorbent material from which the delamination strength test samples are taken is determined from a specimen of the material which is tested according to the conventional Gurley Stiffness test used in the nonwoven, absorbent fiber art. The Gurley Stiffness values of the absorbent material are measured using a Gurley Stiffness Tester (Model No. 4171E), manufactured by Gurley Precision Instruments of Troy, N.Y., U.S.A. The instrument measures the externally applied moment required to produce a given deflection of a test specimen strip of specific dimensions fixed at one end and having a concentrated load applied to the other end. The results are obtained in “Gurley Stiffness” values in units of milligrams. The higher the stiffness of the material, the less flexible, and hence, the less soft, it is.

Tensile Strength

The tensile strength of the absorbent material from which the delamination strength test samples are taken is determined from a specimen of the material which is tested according to the conventional tensile strength test used in the nonwoven, absorbent fiber art. Values for tensile strength of a specified width and length of the material are determined by applying a force longitudinally to the plane of the specimen at a specified constant rate of extension pursuant to the test designated Tensile Strength No. 20.2-89 of the European Disposable And Nonwoven Association (“edana”) and ISO 9073-3:1989 (EN 29073 part 3).

Five (5) elongate specimens of the material are cut with the length parallel to the material machine direction. Each test piece is 50±0.5 mm wide and has a sufficient length to allow jaw separation of 200 mm. The test specimens are conditioned according to ERT 60.1-75. A constant rate of extension of 200 mm/minute is applied by tensile testing machine (dynamometer) holding the test specimen between the tensile machine jaws initially located 200±1 mm apart. The force-elongation curve is recorded. The tensile strength is determined by reading the highest value off of the force-elongation curve.

### EXAMPLES

### Example 1

Strength tests according to the above-described test were conducted on samples of absorbent material having the structure of the material 520 illustrated in FIG. 6. In example 1, the samples were taken from the material 520 as produced by the embodiment of the process illustrated in FIG. 7 except that the water spray addition via the nozzle apparatus 150 was not operated. Thus, no water was added to the material during processing.

The material 520 was produced with (1) an upper layer 529 having a basis weight of 42 gsm (grams per square meter) and 0% superabsorbent polymer (“SAP”), (2) a middle layer 526 having a basis weight of 89 gsm and 47.7% superabsorbent polymer by weight compared to the weight of the pulp fibers in the layer, and (3) a bottom layer 524 having a basis weight of 102 gsm and 45.7% superabsorbent polymer by weight compared to the weight of the pulp fibers in the bottom layer.

In each sample, the top layer 529 and middle layer 526 contained pulp which was 100% southern pine Kraft processed pulp. The bottom layer 524 included southern pine pulp in a blend of 84% by weight of Kraft processed pulp and 16% by weight of cold caustic treated Kraft processed pulp wherein the cold caustic treatment is as defined above with reference to the U.S. patent application Ser. No. 08/370,571 filed Jan. 18, 1995 and as described above with reference to the pulp fibers used in the layers 24, 26, and 28 of the absorbent material 20 shown in FIG. 2.

The superabsorbent material 540 in the Example 1 sample material was in the form of particles sold under the designation No. 7440 by Stockhausen GmbH, Krefeld, Germany and having an office at 2401 Doyle Street, Greensboro, N.C. 27406, U.S.A.

The tissue layer 522 of the material 520 was provided as a web in a roll for use as the web layer 62 in the process shown in FIG. 7. The tissue is sold by Cellu Tissue Corporation, 2 Forbes Street, East Hartford, Conn. 06108, U.S.A. Four different grades of tissue were used to provide various forms of the absorbent material 520: Tissue Grade 3007, Tissue Grade 3007X, Tissue Grade 3007Y, and Tissue Grade 3008. Each tissue grade is produced from 100% southern softwood, and the grades have the characteristics set forth in Table 1A wherein “MD” refers to the tissue manufacturing line machine direction and “CD” refers to the tissue manufacturing line cross direction.

TABLE 1A

| GRADE | | 3007 | 3007X | 3007Y | 3008 |
|---|---|---|---|---|---|
| BASIS WEIGHT | (Lbs./3000 Sq. | 10-10.5 | 10 | 11.7-12.3 | 10.0-11.0 |
| | Ft..) | | | | |
| BULK | (.000″) | — | 76-89 | — | — |
| TENSILE MD | (Grams/Inch) | 475-575 | 496-518 | 475-575 | 250-275 |
| DRY | | | | | |
| TENSILE CD | (Grams/Inch) | 90-125 | 101-105 | 90-125 | 50-60 |
| DRY | | | | | |
| TENSILE MD | (Grams/Inch) | — | 104-118 | — | — |
| WET | | | | | |
| TENSILE CD | (Grams/Inch) | — | — | — | — |
| WET | | | | | |
| ELONGATION | (%) | 12-18 | 24 | 22-28 | 22-28 |
| (@ MD BREAK) | | | | | |
| POROSITY | (Ft³/Min/Ft²) | 200-276 | 273-312 | — | 285 |
| BRIGHTNESS | (Reflectance at | — | 86.4-86.7 | — | 78 |
| | 457 nm) | | | | |
| ABSORBENCY | (Seconds) | — | — | — | 1-9 |
| RATE | | | | | |
| ABSORBENT | (X/Own | — | — | — | — |
| CAPACITY | Weight) | | | | |
| MOISTURE | (%) | — | 7.9 | — | 4.6-6.0 |
| FLUORESCENCE | (None Allowed) | — | — | — | — |

The embodiment of the process illustrated in FIG. 7 was used to produce four (4) runs of the absorbent material 520 illustrated in FIG. 6. each run having a different one of the four grades of tissue listed in Table 1A. The water nozzle apparatus 150 was not operated so that no water was added. The process was operated at a line speed of 90 meters per minute. The upper roll 121 and lower roll 122 were each maintained at a temperature of 150° C. The web was compacted between the rolls 121 and 122 under a load of 315 pounds force per linear inch. The upper roll 121 had a smooth surface, and the lower roll 122 was a “linen” roll with a non-smooth surface.

From each of the four runs, seven (7) specimen samples were prepared and tested according to the above-described Delamination Test Procedure. For each run, the test values of the delamination force (i.e., sample rupture force) were averaged to provide a single, average delamination force. The average delamination force is listed as the “Average Delamination Strength” in the following Table 1B for each of the four runs (designated as Sample Run 1, Sample Run 2, Sample Run 3, and Sample Run 4).

Table 1B also lists the basis weight, density, Gurley Stiffness, and machine direction tensile strength of a specimen of the material in each run.

Table 1B also includes a delamination test force for the average of seven (7) samples of a fifth run, a control run labeled “Control Run 1,” and a delamination test force for the average of seven (7) samples of a sixth run, a control run labeled “Control Run 2.” The Control Run 1 and Control Run 2 material was made by the process described above for the four runs from which the data for Sample Runs 1, 2, 3, and 4 were generated except that the Control Run 1 and Control Run 2 did not use a lower, linen roll 122. Instead, the lower roll 122 for the Control Run 1 and Control run 2 was a smooth surfaced roll like the upper roll 121. The Control Run 1 material included Grade 3007 tissue, and the Control Run 2 material included Grade 3008 tissue. The absorbent core portion composition was identical in Control run 1 and Control Run 2 and was the same as the absorbent core portions of the Sample Runs 1, 2, 3, and 4.

TABLE 1B

| SAMPLE RUN IDENTIFICATION | $\begin{matrix}BASIS \\ WEIGHT \\ ( \frac{grams}{{meter}^{2}} )\end{matrix}$ | $\begin{matrix}DENSITY \\ ( \frac{grams}{{cm}^{3}} )\end{matrix}$ | GURLEY STIFFNESS (milligrams) | MACHINE DIRECTION TENSILE STRENGTH (newtons) | TISSUE GRADE | AVERAGE DELAMINATION STRENGTH OF SEVEN SAMPLES (newtons) | COMMENTS |
|---|---|---|---|---|---|---|---|
| Sample Run 1 | 247 | 0.27 | 164.6 | 11.53 | 3007 | 2.61 | Tissue pulled off |
| | | | | | | | easily. |
| Sample Run 2 | 250 | 0.27 | 210.4 | 12.66 | 3007X | 4.52 | Tissue pulled off |
| | | | | | | | pulling some |
| | | | | | | | fibers from core. |
| Sample Run 3 | 260 | 0.28 | 203.9 | 16.29 | 3007Y | 5.78 | Tissue pulled lots |
| | | | | | | | of fibers and |
| | | | | | | | SAP. |
| Sample Run 4 | 245 | 0.26 | 181.1 | 12.16 | 3008 | 5.12 | Tissue pulled off |
| | | | | | | | pulling some |
| | | | | | | | fibers from core. |
| Control Run 1 | 251 | 0.29 | 192 | 11.43 | 3007 | 2.66 | Tissue pulled off |
| (Smooth Rolls) | | | | | | | easily. |
| Control Run 2 | 250 | 0.27 | 180 | 11.70 | 3008 | 2.17 | Tissue pulled off |
| (Smooth Rolls) | | | | | | | easily. |

The values in Table 1B show force values for the delamination strength at which the tissue pulled off of the absorbent core (i.e., the separation of tissue layer 522 from the absorbent core portion bottom layer 524). These values are for samples of material to which no water was added during the manufacturing process (i.e., the water addition apparatus 150 in FIG. 7 was not operated). The test results show that the various grades of tissue pulled off of the absorbent core portion at delamination forces of less than 6 newtons.

It should also be noted that the delamination strength can be increased by the use of a non-smooth roll or linen roll compared to a smooth roll. For example, in the Control Run 2 (using smooth rolls), the delamination strength was only 2.17 newtons for a web with Grade 3008 tissue, but was much greater (i.e., 5.12 newtons) for the web with Grade 3008 tissue in Sample Run 4 (using the lower linen roll with a non-smooth surface).

Thus, for some combination of tissue and core processed with the linen roll, the use of the non-smooth roll increases the delamination strength of the tissue/core bond. This increase in delamination strength occurs even without the addition of moisture—the effect of moisture addition per se being described in detail in Example 2 immediately below.

### Example 2

The effect of moisture (e.g., water) addition to the tissue during the process of making the absorbent material was investigated. In Example 2, the embodiment of the process illustrated in FIG. 7 was operated as in Example 1 described above, but with water sprayed against the tissue layer web for runs using three different tissues, and without water sprayed against the tissue for two other “control runs.” Table 2 lists characteristics of the material produced in each run in Example 2.

The upper layer 529, middle layer 526, and bottom layer 524 of the absorbent core portion 536 for the Example 2 runs had the same pulp and superabsorbent composition as in Example 1. The processing line (FIG. 7) was run at the same speed and same calender roll temperature and compression force as in Example 1. For Sample Runs 1, 3, and 4 in Table 2, water was sprayed from the above-described nozzles of the device 150 (FIG. 7) against the bottom of the tissue layer web 62 from a distance 10 inches below the web 62. Six nozzles were spaced apart on 30 cm centers across the width of the web. The water was supplied to the nozzles at a pressure of 15 psig and a temperature of 15° C. at a rate to produce a 3% moisture addition based upon the weight of the absorbent material web prior to the moisture addition.

Immediately after the web passed through the calendering rolls, the moisture content of the web was low. However, after the samples had come to equilibrium with ambient atmosphere, measurement of the moisture in the material in each Sample Run 1, 3, and 4 showed the moisture content to be within the range of from 4% to 4.5% by weight based upon the total weight of the sample with the moisture included.

For the Control Run 1 and Control Run 2 listed in Table 2, both calender rolls 121 and 122 were smooth.

For the Sample Runs 1, 3, and 4 in Table 2, the lower roll 122 was a knurled, linen roll, and the upper roll 121 was smooth.

The identification of the grades of tissue used in the Example 2 runs listed in Table 2 corresponds to the Example 1 tissue grades listed in Table 1A above.

For the Control Run 2 samples tested to determine the Average Delamination Strength in Table 2, the tissue was first manually removed in the laboratory prior to testing so that only the absorbent core portion was tested for delamination strength.

Owing to lack of available time in a production facility, Sample Run 2 using Grade 3007X tissue could not be produced. Thus, Table 2 does not list a Sample Run 2.

TABLE 2

| SAMPLE RUN IDENTIFICATION | $\begin{matrix}BASIS \\ WEIGHT \\ ( \frac{grams}{{meter}^{2}} )\end{matrix}$ | $\begin{matrix}DENSITY \\ ( \frac{grams}{{cm}^{3}} )\end{matrix}$ | GURLEY STIFFNESS (milligrams) | MACHINE DIRECTION TENSILE STRENGTH (newtons) | TISSUE GRADE | AVERAGE DELAMINATION STRENGTH OF SEVEN SAMPLES (newtons) | COMMENTS |
|---|---|---|---|---|---|---|---|
| Sample Run 1 | 253 | 0.29 | 185.7 | 17.52 | 3007 | 7.78 | Core delaminated |
| | | | | | | | instead of tissue |
| | | | | | | | pulling from |
| | | | | | | | core. |
| Sample Run 3 | 260 | 0.29 | 217.1 | 17.87 | 3007Y | 8.40 | Core delaminated |
| | | | | | | | instead of tissue |
| | | | | | | | pulling from |
| | | | | | | | core. |
| Sample Run 4 | 255 | 0.29 | 195.0 | 14.69 | 3008 | 7.86 | Core delaminated |
| | | | | | | | instead of tissue |
| | | | | | | | pulling from |
| | | | | | | | core. |
| Control Run 1 | 251 | 0.29 | 192 | 11.43 | 3007 | 2.66 | Tissue pulled |
| (Smooth Rolls) | | | | | | | from core. |
| Control Run 2 | 237 | 0.27 | — | — | 3007 | 3.42 | Core delaminated |
| (Smooth Rolls) | | | | | | | (the tissue had |
| | | | | | | | been manually |
| | | | | | | | removed from |
| | | | | | | | samples prior to |
| | | | | | | | test). |

Table 1B and Table 2 can be compared to show the effects of the moisture addition. The bond strength between the tissue and absorbent core portion improves when the moisture is added. Table 1B shows that in the Sample Runs 1, 2, 3, and 4 where no water was added, the tissue pulled away from the core before the core could delaminate. On the other hand, Table 2 shows that in Sample Runs 1, 3, and 4 where water was added, the tissue was so strongly bonded that it did not pull away, and the core delaminated instead.

Table 2 also shows that there is a significant improvement in the integrity of the absorbent core portion per se (portion 536 in FIG. 6) when water is added during the process illustrated in FIG. 7. The Control Run 2 in Table 2 was produced without water addition, and the tissue was manually removed in the laboratory before the delamination test was conducted on samples of Control Run 2. The Control Run 2 sample cores ruptured or delaminated with an average delamination strength of 3.42 newtons. This is considerably less than the average delamination strength of the samples tested for Sample Run 1, Sample Run 3, and Sample Run 4 wherein the web was processed with the addition of water and where the core ruptured or delaminated at average strength values in excess of 7 newtons (considerably higher than the core delamination strength value of 3.42 newtons for the Control Run 2 sample average).

### Example 3

Example 3 investigated the effect of spraying water onto tissue on a laboratory scale. Absorbent material having the structure illustrated in FIG. 6 was used, but the composition of the absorbent material differed from that described above in Example 1 and in Example 2. Specifically, the material 520 (FIG. 6) used in Example 3 was produced with an upper layer 529 having a basis weight of 25 gsm and 0% superabsorbent polymer, a middle layer 526 having a basis weight of 225 gsm and 57% superabsorbent polymer by weight compared to the weight of the pulp fibers in the layer, and a bottom layer 524 having a basis weight of 233 gsm and 54% superabsorbent polymer by weight compared to the weight of the pulp fibers in the bottom layer. In each sample, the top layer 529 and the middle layer 526 contained pulp which was 100% southern pine Kraft processed pulp. The bottom layer 524 included southern pine pulp in a blend of 78% by weight of Kraft processed pulp and 22% by weight of cold caustic treated Kraft processed pulp wherein the cold caustic treatment is as defined above with reference to the U.S. patent application Ser. No. 08/370,571, filed Jan. 18, 1995 and as described above with reference to the pulp fibers used in the layers 24, 26, and 28 of the absorbent material 20 shown in FIG. 2. The superabsorbent material 540 in the Example 3 sample material was in the form of particles sold by Stockhausen under the above-described designation No. 7440. The tissue layer 522 (FIG. 6) was Grade 3008 (Table 1A above).

Absorbent material for Example 3 was prepared on a processing line as diagrammatically illustrated in FIG. 7, but without the operation of the water addition device 150 so as to prevent water addition to the formed web. The processing line was run at a speed of 35 meters per minute. The upper roll 121 and lower roll 122 were each maintained at a temperature of 140° C. Both the upper roll 121 and the lower roll 122 were smooth surfaced and the web was compacted between the rolls under a load of 315 pounds force per linear inch. After the absorbent material was formed, a sample of the formed material was taken, and the tissue layer was manually removed from the sample. A new piece of tissue was then placed on the absorbent core (tissue Grade 3008 in Table 1A).

In a first sample, Sample 1 of Example 3, the new tissue, without water addition, was bonded to the core in a heated press at 300° F. and a pressure of 120 psi for 60 seconds. No water was added.

A second sample, Sample 2, was prepared in a manner identical with Sample 1, except that before Sample 2 was placed in the heated press, the new tissue on the Sample 2 was sprayed with water to increase the moisture to 4% by weight of the oven dry weight of the sample. The wetted Sample 2 was then pressed in the heated press under the same conditions as Sample 1.

A third sample, Sample 3, was prepared in the same manner as Sample 2 described above, except that the amount of water added was 11% by weight of the oven dry weight of the sample.

Finally, a Control Sample was prepared, and that consisted only of an absorbent core material produced on the FIG. 7 processing line in the same way as the material used to make Samples 1 and 2 except that the material for the Control Sample was produced without water addition, and then the tissue was removed prior to delamination testing.

Table 3 presents the results of delamination strength tests of the samples. The higher standard deviation for Sample 2 is due to non-uniformity in the spraying of water in the laboratory spray system. This results in variability in bonding between the tissue and core. The Table 3 data shows that the addition of moisture improves bonding of the absorbent core portion to the tissue.

TABLE 3

| | | AVERAGE | |
|---|---|---|---|
| | | DELAMINATION | |
| | WATER | STRENGTH OF | |
| | ADDITION | SEVEN SAMPLES | |
| SAMPLE | QUANTITY | (newtons) | COMMENTS |
| Control | 0% Water | 2.26, std. dev. = 0.52 | No tissue was |
| Sample | | | used. Core |
| | | | delaminated |
| Sample 1 | 0% Water | 0 | Tissue was not |
| | | | bonded to core at |
| | | | all |
| Sample 2 | 4% Water | 4.80, std. dev. = 1.55 | Tissue pulled |
| | | | away from core |
| Sample 3 | 11% Water | — | Tissue well |
| | | | bonded with core |

In case of Sample 1, there was very little bonding between the core and tissue. In case of Sample 3, the tissue was bonded so well with the core that the core disintegrated first, and the greater force or strength of the bond between the tissue and core could not be measured. However, there is a general trend toward substantial improvements in bonding by the addition of water.

### Example 4

The type of laboratory investigation described in Example 3 was conducted in Example 4 on absorbent material having the same structure illustrated in FIG. 6 and the same layer composition (i.e., percent superabsorbent, percent Kraft pulp and cold caustic treated pulp) as employed in the material in Examples 1 and 2. The absorbent material from which the Example 4 test samples were taken was initially produced in the same manner as in Example 3 described above.

However, a control sample of an absorbent core was prepared from absorbent material produced on the processing line illustrated in FIG. 7 without water addition, and then the tissue was removed from the core. This control sample is identified in Table 4 below as the “Control Sample.”

Sample 1 was prepared from an absorbent core with the old tissue (Grade 3008) manually removed. A new tissue (Grade 3008) was placed on the core, and the assembly was pressed in a heated press at 300° F. and 120 psi for 60 seconds. No water was added.

A second sample, Sample 2 was prepared from an absorbent core with the old tissue (Grade 3008) removed. New tissue (Grade 3008) was placed on the absorbent core, and water was sprayed on the tissue side of the core to increase the moisture content to 11% by weight compared to the oven dry weight of the core. The assembly was then heat pressed in the same manner as the Example 4, Sample 1 described above.

The samples were then tested for delamination strength, and the results are set forth in Table 4 below.

TABLE 4

| | | AVERAGE | |
|---|---|---|---|
| | | DELAMINATION | |
| | WATER | STRENGTH OF | |
| | ADDITION | SEVEN SAMPLES | |
| SAMPLE | QUANTITY | (newtons) | COMMENTS |
| Control | 0% Water | 2.98, std. dev. = 0.53 | No tissue was |
| Sample | | | used. Core |
| | | | delaminated |
| Sample 1 | 0% Water | 0 | Tissue was not |
| | | | bonded to core |
| | | | at all |
| Sample 2 | 11% Water | 7.00, std. dev. = 2.7 | Tissue pulled |
| | | | away from core |

Table 4 shows that the delamination strength of the material without water addition was lower than the delamination strength of the material when water is added.

In case of Sample 1, there was very little bonding between the core and tissue. Also, due to the limitations of the laboratory spray system, it was not possible to add a lower dosage of water than 11%. Hence, the 11% charge was used. The higher standard deviation for Sample 2 is due to non-uniformity in the spraying of water. This results in some variability in bonding between the tissue and core. This data shows that the addition of moisture improves bonding of the absorbent core portion to the tissue.

Affect of Moisture Addition on Absorbent Material

As described above in the section entitled “PRODUCTION PROCESS AND APPARATUS: FIRST EMBODIMENT,” moisture (e.g., steam or water spray) can be added to the absorbent material to effect the establishment of hydrogen bonding of pulp fibers to each other within the core portion (pulp layers), as well as of the tissue layer to the core portion pulp fibers, so as to increase the strength and integrity of the finished absorbent material. It has been discovered that, contrary to conventional expectations, the moisture addition does not significantly increase the stiffness of the finished material. The production of a finished absorbent material with increased integrity and strength from hydrogen bonding, but without increased stiffness, is highly desirable where the material is to be used in absorbent products such as disposable diapers and feminine hygiene products which should be soft and flexible.

Samples of absorbent material were tested for strength and stiffness. Some of the samples that were tested did not have moisture added, and some of the samples were tested after the addition of moisture. The test results are presented in Table 5 below.

Table 5 lists the values for the Gurley Stiffness and Machine Direction Tensile Strength which were determined by testing various test specimens of the Sample A material, the Sample B material, and the Control material. For each of the Sample A, Sample B, and Control materials, six test specimens were tested for the Gurley Stiffness values, and the average value was calculated and listed in Table 5. For each of the Sample A, Sample B, and Control materials, five specimens were tested for the Machine Direction Tensile Strength, and the average value was calculated and listed in Table 5. The Gurley Stiffness test was conducted as set forth above in the section entitled “GURLEY STIFFNESS DETERMINATION,” and the Machine Direction Tensile Strength test was conducted as set forth above in the section entitled “TENSILE STRENGTH.”

The Sample A material, Sample B material, and Control material listed in Table 5 were initially taken from absorbent material produced according to the process described in Example 3 set forth above. Thus, no water spray was employed in the initial production of the material used for Table 5 tests. However, unlike in Example 3, the tissue layer was not manually removed from the finished production material used for the Table 5 tests.

The Table 5 Control specimens were taken from the production material without further processing and were tested to determine the Gurley Stiffness values and Machine Direction Tensile Strength values listed in Table 5.

For the Table 5 Sample A specimens, before conducting the stiffness and strength tests, water was sprayed against each specimen top pulp layer (i.e., upper layer 529 in FIG. 6) to increase the moisture content to 5% by weight of the initial dry specimen weight. The Sample A specimens were then pressed in a heated flat press at 300° F. and a pressure of 120 psi for 30 seconds.

For the Table 5 Sample B specimens, before conducting the stiffness and strength tests, a new, additional layer of tissue was applied to the top pulp layer (i.e., upper layer 529 in FIG. 6), and then the exposed surface of the new tissue layer on each specimen was sprayed with water to increase the moisture content to 5% by weight of the initial dry specimen weight. The additional tissue applied to the top layer was the same type of tissue as on the bottom layer (i.e., tissue Grade 3308 identified in Table 1A as described above in reference to Example 3). The Sample B specimens were then pressed in a heated flat press at 300° F. and a pressure of 120 psi for 30 seconds.

For each of the Sample A, Sample B, and Control materials, six specimens were also measured to determine the basis weight and density, and the average values of the basis weight and of the density were calculated and listed in Table 5. For the Sample A and Sample B materials, the basis weight and density were determined after the specimens were removed from the heated press. The basis weights listed in Table 5 were determined as described above in the section entitled “BASIS WEIGHT DETERMINATION.” The densities listed in Table 5 were determined as described above in the section entitled “DENSITY DETERMINATION.”

Table 5 shows that the average value of the tensile strength of the Control material (to which no water was added) was 15.57 newtons. Compared with the Sample A and Sample B average tensile strength values of 25.42 newtons and 27.25 newtons, respectively, the 15.57 newtons strength of the Control material is considerably less. The tensile strength of the Sample A and B material to which moisture was added is at least about 63% greater than the tensile strength of the Control material to which no moisture was added. Moreover, and unexpectedly, the Gurley Stiffness of the Sample A and B material did not increase to any significant extent compared to the Control material. The increase was less than 0.01% for Sample B, and the increase was less than 3.5% for Sample A (which included the second, top layer of tissue). Thus, the addition of moisture facilitated the creation of hydrogen bonds which increased the material strength without undesirably increasing the stiffness. Even in the Sample B material specimens, to which an extra (second) layer of tissue was added, the stiffness is not significantly increased compared to the Control material.

TABLE 5

| SAMPLE IDENTIFICATION | $\begin{matrix}BASIS \\ WEIGHT \\ ( \frac{grams}{{meter}^{2}} )\end{matrix}$ | $\begin{matrix}DENSITY \\ ( \frac{grams}{{cm}^{3}} )\end{matrix}$ | GURLEY STIFFNESS (milligrams) | MACHINE DIRECTION TENSILE STRENGTH (newtons) |
|---|---|---|---|---|
| Sample A | 532 | 0.41 | 1253 | 25.42 |
| Sample B | 530 | 0.39 | 1213 | 27.25 |
| Control | 552 | 0.36 | 1212 | 15.57 |

It will be readily apparent from the foregoing detailed description of the invention and from the illustrations thereof that numerous variations and modifications may be effected without departing from the true spirit and scope of the novel concepts or principles of the invention.

What is claimed is:

1. A process for making an absorbent material comprising the steps of:
(A) providing a carrier layer;
(B) forming a web having at least one layer of a mixture of cellulosic fibers and superabsorbent material on top of said carrier layer wherein at least some of said cellulosic fibers are provided by
(1) treating a liquid suspension of pulp containing said cellulosic fibers at a temperature of from about 15° C. to about 60° C. with an aqueous alkali metal salt solution having an alkali metal salt concentration of from about 2 weight percent to about 25 weight percent of said solution for a period of time ranging from about 5 minutes to about 60 minutes, and
(2) blending said cellulosic fibers and said superabsorbent material with controlled air circulation and mechanical agitation to form said mixture of cellulosic fibers and superabsorbent material free of chemical binding agents and heat set bonding agents;
(C) forming a layer of said cellulosic fibers which is free of said superabsorbent material and which is disposed on top of said at least one layer;
(D) increasing the moisture content of said web to increase the web density by conveying said web through a region into which steam having a temperature of above about 100° C. is discharged and maintaining the residence time of the web in said region for at least about 0.1 second;
(E) after step (D), compacting said web to less than about 3 mm at an elevated temperature to further increase the web density, and to increase bonding within the web, said step of compacting said web including the step of compacting said web between two calendering rolls wherein (1) at least one of the rolls is heated to a surface temperature in the range of between about 70° C. to about 200° C., (2) at least the roll contacting said carrier layer has a non-smooth surface to embed portions of said carrier layer in said at least one layer of a mixture of cellulosic fibers and superabsorbent material, and (3) the rolls exert a load of at least about 28 newtons per millimeter (160 pounds force per inch) of transverse web width; and
(F) after said web cools to ambient temperature, maintaining the moisture content in said web in equilibrium with the ambient atmosphere at between about 3% and about 8% by weight based upon the total weight of said web with said moisture.

2. The process in accordance with claim 1 in which
step (A) includes providing said carrier layer as a tissue layer; and
step (B)(1) includes one of the following steps (a) and (b) prior to step (B)(2): (a) flash drying said cellulosic fibers, and (b) processing said cellulosic fibers through a hammermill.

3. A process for making an absorbent material comprising the steps of:
(A) forming a web having at least one layer of a mixture of cellulosic fibers and superabsorbent material free of added chemical binders and heat set bonding agents;
(B) increasing the moisture content of said web by adding between about 1% and 8% moisture, based upon the total weight of the web prior to moisture addition, to increase the web density; and
(C) after step (B), compacting said web at an elevated temperature to further increase the web density.

4. The process in accordance with claim 3 in which step (A) includes forming said at least one layer of cellulosic fibers and superabsorbent material on a carrier layer which becomes part of said web.

5. The process in accordance with claim 4 in which step (A) includes air laying a blend of said cellulosic fibers and superabsorbent material on said carrier layer comprising one of a layer of regular tissue or a layer of crepe tissue.

6. The process in accordance with claim 4 in which step (A) includes air laying a blend of said cellulosic fibers and superabsorbent material on said carrier layer wherein said carrier layer comprises about 3 weight percent to about 20 weight percent of the combined weight said absorbent material and carrier layer.

7. The process in accordance with claim 3 further including, before step (A), the steps of
(1) treating a liquid suspension of pulp containing said cellulosic fibers at a temperature of from about 15° C. to about 60° C. with an aqueous alkali metal salt solution having an alkali metal salt concentration of from about 2 weight percent to about 25 weight percent of said solution for a period of time ranging from about 5 minutes to about 60 minutes, and
(2) blending said cellulosic fibers and said superabsorbent material with controlled air circulation and mechanical agitation to form said mixture of cellulosic fibers and superabsorbent material.

8. The process in accordance with claim 7 in which
said step (A) includes forming said web with from about 10 weight percent to about 60 weight percent superabsorbent material based upon the total weight of said web with said superabsorbent material; and
said step (1) includes one of the following steps (a) and (b) prior to said step (2): (a) flash drying said cellulosic fibers, and (b) processing said cellulosic fibers through a hammer mill.

9. The process in accordance with claim 3 in which step (A) includes forming a layer of said cellulosic fibers which is free of said superabsorbent material and which is disposed on top of said at least one layer.

10. The process in accordance with claim 3 in which step (A) includes forming said at least one layer containing less than about 10 weight percent moisture based upon the total weight of said one layer with said moisture.

11. The process in accordance with claim 3 in which step (A) includes forming said at least one layer with a water content of less than about 7 weight percent based upon the total weight of said one layer with said moisture.

12. The process in accordance with claim 3 in which step (A) includes supporting said web on a moving screen.

13. The process in accordance with claim 3 in which step (A) includes air laying said mixture of cellulosic fibers and superabsorbent material to form said at least one layer.

14. The process in accordance with claim 3 in which step (B) includes moving said web through a controlled atmosphere to increase the moisture content of said web to a level of less than about 10 weight percent based upon the total weight of said web with said moisture.

15. The process in accordance with claim 14 in which step (B) includes injecting low pressure steam into an enclosure adjacent one side of said web.

16. The process in accordance with claim 15 in which step (B) includes maintaining said steam in said enclosure on said one side of said web at a temperature of at least about 100° C. and at a pressure of at least about 1.01325×10⁵ Pascal while moving said web at a speed to provide a residence time in said enclosure of at least about 0.1 second.

17. The process in accordance with claim 3 in which step (C) includes compacting said web between at least two calendering rolls.

18. The process in accordance with claim 17 in which step (C) includes maintaining at least one of said rolls at an elevated temperature.

19. The process in accordance with claim 18 in which step (C) includes maintaining said at least one roll at a temperature above at least about 70° C.

20. The process in accordance with claim 18 in which step (C) includes maintaining said at least one roll at a temperature above at least about 80° C.

21. The process in accordance with claim 17 in which
step (A) includes forming said at least one layer of cellulosic fibers and absorbent material on a carrier layer which becomes part of said web; and
step (C) includes providing one of said rolls with a smooth surface and the other of said rolls with a knurled surface having a depth of greater than 5% of the thickness of said carrier layer.

22. The process in accordance with claim 18 in which step (C) includes maintaining both of said rolls at an elevated temperature.

23. The process in accordance with claim 17 in which
step (A) includes air laying a blend of said cellulosic fibers and superabsorbent material to form said at least one layer of said cellulosic fibers and superabsorbent material on a layer of tissue; and
step (C) includes providing a knurled surface on the roll contacting said carrier layer so as to embed portions of said carrier layer in said at least one layer of a mixture of cellulosic fibers and superabsorbent material.

24. The process in accordance with claim 17 in which step (C) includes compacting said web between said rolls under a load of between about 28 and about 400 newtons per millimeter of transverse web width.

25. The process in accordance with claim 3 further including the additional step, before step (B), of forming a layer of exclusively superabsorbent material adjacent said at least one layer of a mixture of said cellulosic fibers and superabsorbent material.

26. A process for making an absorbent material comprising the steps of:
(A) forming a web having a carrier layer on which is disposed at least one absorbent layer containing cellulosic fibers free of added chemical binders and heat set bonding agents;
(B) spraying water against said web to add between about 1% and 8% moisture by weight based upon the weight of said web prior to moisture addition; and
(C) after step (B), compacting said web at an elevated temperature to further increase the web density and to effect either an increase in the strength of said at least one absorbent layer and/or an increase in the bonding strength of said carrier layer to said at least one absorbent layer.

27. The process in accordance with claim 26 in which step (A) includes forming said at least one layer of cellulosic fibers with superabsorbent particles which becomes part of said at least one absorbent layer.

28. The process in accordance with claim 27 in which step (A) includes air laying a blend of said cellulosic fibers and superabsorbent material on said carrier layer comprising a layer of regular tissue or a layer of crepe tissue.

29. The process in accordance with claim 26 further including, before step (A), the steps of
(1) treating a liquid suspension of pulp containing said cellulosic fibers at a temperature of from about 15° C. to about 60° C. with an aqueous alkali metal salt solution having an alkali metal salt concentration of from about 2 weight percent to about 25 weight percent of said solution for a period of time ranging from about 5 minutes to about 60 minutes, and
(2) blending said treated cellulosic fiber pulp with untreated cellulosic fiber pulp to form a blend.

30. The process in accordance with claim 29 in which
said step (A) includes forming said web with from about 10 weight percent to about 60 weight percent superabsorbent material based upon the total weight of said web with said superabsorbent material; and
said step (1) includes one of the following steps (a) and (b) prior to said step (2): (a) flash drying said cellulosic fibers, and (b) processing said cellulosic fibers through a hammer mill.

31. The process in accordance with claim 26 in which step (A) includes forming a layer of said cellulosic fibers which is free of said superabsorbent material and which is disposed on top of said at least one layer.

32. The process in accordance with claim 26 in which step (B) includes adding 3% moisture to said web based upon the total weight of said web prior to moisture addition.

33. The process in accordance with claim 32 further including the step, after step (C), of letting said web reach equilibrium with ambient atmosphere to produce a web in which the moisture content is between about 3% and about 8% based upon the total weight of said web with said moisture after equilibrium with the ambient atmosphere has been achieved.

34. The process in accordance with claim 26 in which step (A) includes supporting said web on a moving screen.

35. The process in accordance with claim 26 in which step (A) includes air laying said cellulosic fibers to form said at least one layer.

36. The process in accordance with claim 26 in which step (B) includes spraying water at the top side of said at least one absorbent layer or the bottom side of said carrier layer.

37. The process in accordance with claim 36 in which step (B) includes spraying said water at a pressure of about 15 psig from a plurality of spaced apart nozzles which each have a conical spray pattern while moving said web at a speed of between about 30 to about 300 meters per minute.

38. The process in accordance with claim 26 in which step (C) includes compacting said web between two calendering rolls.

39. The process in accordance with claim 38 in which step (C) includes maintaining at least one of said rolls at an elevated temperature.

40. The process in accordance with claim 39 in which step (C) includes maintaining said both rolls at temperatures in the range between about 70° C. and about 200° C.

41. The process in accordance with claim 39 in which step (C) includes providing one of said rolls with a smooth surface and the other of said rolls with a surface having indentations with a depth of greater than 5% of the thickness of said carrier layer so as to contact said carrier layer so as to embed portions of said carrier layer in said at least one absorbent layer.

42. The process in accordance with claim 38 in which step (C) includes compacting said web between said rolls under a load of between about 160 and about 2284 pounds force per inch of transverse web width.

43. An absorbent material which
(I) is made by the process comprising the steps of:
(A) forming a web having a layer of tissue on which is disposed at least one absorbent layer containing cellulosic fibers free of added chemical binders and heat set bonding agents;
(B) spraying water against said layer of tissue to increase the moisture content of said web to between about 1% and about 8% by weight based upon the weight of the web prior to moisture addition; and
(C) after step (B), compacting said web at an elevated temperature to further increase the web density and to effect an increased bonding of said tissue to said at least one absorbent layer; and
(II) has a bond strength between said layer of tissue and said at least one absorbent layer that exceeds a test delamination force of 3 newtons.

44. The absorbent material in accordance with claim 43 in which said cellulosic fibers include fibers which have been processed by one of the following steps (a) and (b): (a) flash drying said cellulosic fibers, and (b) processing said cellulosic fibers through a hammermill.

45. The absorbent material in accordance with claim 43 in which said tissue layer comprises about 3 weight percent to about 20 weight percent of the combined weight of said absorbent layer and tissue layer.

46. The absorbent material in accordance with claim 43 in which some of said cellulosic fibers are made for use in said absorbent layer by first treating a liquid suspension of pulp containing said cellulosic fibers at a temperature of from about 15° C. to about 60° C. with an aqueous alkali metal salt solution having an alkali metal salt concentration of from about 2 weight percent to about 25 weight percent of said solution for a period of time ranging from about 5 minutes to about 60 minutes.

47. The absorbent material in accordance with claim 43 in which said at least one absorbent layer includes from about 10 weight percent to about 60 weight percent superabsorbent material based upon the total weight of said layer with said superabsorbent material.

48. The absorbent material in accordance with claim 43 in which said material includes an absorbent layer of said cellulosic fibers which is free of superabsorbent material and which is disposed on top of said at least one absorbent layer.

49. The absorbent material in accordance with claim 43 in which step (B) includes spraying water at the bottom side of said layer of tissue.

50. The absorbent material in accordance with claim 43 in which step (B) includes spraying said water at a pressure of about 15 psig from a plurality of spaced apart nozzles which each have a conical spray pattern while moving said web at a speed of between about 30 to about 300 meters per minute.

51. The absorbent material in accordance with claim 43 in which step (C) includes compacting said web between two calendering rolls.

52. The absorbent material in accordance with claim 51 in which step (C) includes maintaining at least one of said rolls at an elevated temperature.

53. The absorbent material in accordance with claim 52 in which step (C) includes maintaining both of said rolls at temperatures in the range between about 70° C. and about 200° C.

54. The process in accordance with claim 51 in which step (C) includes providing one of said rolls with a smooth surface and the other of said rolls with a non-smooth surface defining indentations having a depth of greater than 5% of the thickness of said layer of tissue so as to contact said tissue so as to embed portions of said tissue in said at least one absorbent layer.

55. The process in accordance with claim 51 in which step (C) includes compacting said web between said rolls under a load of between about 160 and about 2284 pounds force per inch of transverse web width.

56. The absorbent material in accordance with claim 43 in which said absorbent material includes three of said absorbent layers arranged as a top absorbent layer, a middle absorbent layer, and a bottom absorbent layer adjacent said layer of tissue.

57. The absorbent material in accordance with claim 56 in which
said top absorbent layer has a basis weight of about 42 grams per square meter and no superabsorbent material;
said middle absorbent layer has a basis weight of about 87 grams per square meter and about 47% superabsorbent material; and
said bottom absorbent layer has a basis weight of about 102 grams per square meter and about 45% superabsorbent material.

58. The absorbent material in accordance with claim 56 in which said top absorbent layer and middle absorbent layer contain pulp which is 100% southern pine Kraft processed pulp.

59. The absorbent material in accordance with claim 56 in which said bottom absorbent layer includes southern pine pulp in a blend of 74% by weight of Kraft processed pulp and 16% by weight of cold caustic treated Kraft processed pulp.

60. The absorbent material in accordance with claim 43 in which said tissue layer comprises tissue produced from substantially 100% soft wood and having (a) a basis weight of between about 10 and about 13 pounds per 3,000 square feet, (b) a dry tensile strength in the machine direction of between about 275 and about 575 grams per inch, (c) a dry tensile strength in the cross direction of between about 60 and about 125 grams per inch, and (d) a porosity of between about 200 and about 312 cubic feet per minute per square foot.

61. The absorbent material in accordance with claim 43 in which said layer of tissue is a first layer of tissue, and said web includes a second layer of tissue on a side of said web facing away from said first layer of tissue.

62. An absorbent material which is made by the process comprising the steps of:
(A) forming a web having a layer of tissue on which is disposed at least one absorbent layer containing cellulosic fibers free of added chemical binders and heat set bonding agents;
(B) applying water to said web to increase the moisture content of said web to between about 1% and about 8% by weight based upon the weight of the web prior to moisture addition; and
(C) after step (B), compacting said web at an elevated temperature to further increase the web density and to increase the web tensile strength of the material by at least 60% without increasing the Gurley Stiffness more than 0.01% compared to making the material by omitting step (B).

63. An absorbent material comprising:
a web having a carrier layer on which is disposed at least one absorbent layer containing at least cellulosic fibers free of added chemical binders and heat set bonding agents;
said web having a density between about 0.25 grams per cubic centimeter and about 0.5 grams per cubic centimeter;
said web having a basis weight between about 150 grams per square meter and about 600 grams per square meter;
said web having a Gurley Stiffness of less than about 1500 milligrams;
said web having a machine direction tensile strength greater than about 9 newtons; and
said web having a bond strength between said at least one absorbent layer and said carrier layer that exceeds a test delamination force of 3 newtons.

64. The absorbent material in accordance with claim 63 in which the absorbent material is made by the process comprising the steps of:
(A) forming said web having said carrier layer comprising a layer of tissue on which is disposed said at least one absorbent layer;
(B) spraying water against said layer of tissue to increase the moisture content of said web to between about 1% and about 8% by weight based upon the weight of the web prior to moisture addition; and
(C) after step (B), compacting said web at an elevated temperature to further increase the web density and to effect an increased bonding of said layer of tissue to said at least one absorbent layer.

65. An absorbent material in accordance with claim 63 in which the absorbent material is made by the process comprising the steps of:
(A) forming a web having said carrier layer of tissue on which is disposed said at least one absorbent layer; and
(B) after step (A), compacting said web at an elevated temperature to further increase the web density and to effect an increased bonding of said layer of tissue to said at least one absorbent layer, said step of compacting including compacting said web between a pair of rolls wherein one of the rolls is in contact with said layer of tissue and has surface indentations.

66. The absorbent material in accordance with claim 63 in which said cellulosic fibers include fibers which have been processed by one of the following steps (a) and (b): (a) flash drying said cellulosic fibers, and (b) processing said cellulosic fibers through a hammermill.

67. The absorbent material in accordance with claim 63 in which said carrier layer comprises a tissue layer which is about 3 weight percent to about 20 weight percent of the combined weight of said absorbent layer and tissue layer.

68. The absorbent material in accordance with claim 63 in which some of said cellulosic fibers are made for use in said absorbent layer by first treating a liquid suspension of pulp containing said cellulosic fibers at a temperature of from about 15° C. to about 60° C. with an aqueous alkali metal salt solution having an alkali metal salt concentration of from about 2 weight percent to about 25 weight percent of said solution for a period of time ranging from about 5 minutes to about 60 minutes.

69. The absorbent material in accordance with claim 63 in which said at least one absorbent layer includes from about 10 weight percent to about 60 weight percent superabsorbent material based upon the total weight of said at least one absorbent layer with said superabsorbent material.

70. The absorbent material in accordance with claim 63 in which said web includes an absorbent layer of said cellulosic fibers which is free of superabsorbent material and which is disposed on top of said at least one absorbent layer.

71. The absorbent material in accordance with claim 64 in which step (B) includes spraying water at the bottom side of said carrier layer of tissue.

72. The absorbent material in accordance with claim 64 in which step (B) includes spraying said water at a pressure of about 15 psig from a plurality of spaced apart nozzles which each have a conical spray pattern while moving said web at a speed of between about 30 to about 300 meters per minute.

73. The absorbent material in accordance with claim 64 in which step (C) includes compacting said web between two calendering rolls.

74. The absorbent material in accordance with claim 73 in which step (C) includes maintaining at least one of said rolls at an elevated temperature.

75. The absorbent material in accordance with claim 74 in which step (C) includes maintaining both of said rolls at temperatures in the range between about 70° C. and about 200° C.

76. The process in accordance with claim 73 in which step (C) includes providing one of said rolls with a smooth surface and the other of said rolls with a knurled surface having a depth of greater than 5% of the thickness of said layer of tissue so as to contact said tissue so as to embed portions of said tissue in said at least one absorbent layer.

77. The process in accordance with claim 73 in which step (C) includes compacting said web between said rolls under a load of between about 160 and about 2284 pounds force per inch of transverse web width.

78. The absorbent material in accordance with claim 63 in which said absorbent material includes three of said absorbent layers arranged as a top absorbent layer, a middle absorbent layer, and a bottom absorbent layer adjacent said layer of tissue.

79. The absorbent material in accordance with claim 78 in which
said top absorbent layer has a basis weight of about 42 grams per square meter and no superabsorbent material;
said middle absorbent layer has a basis weight of about 87 grams per square meter and about 47% superabsorbent material; and
said bottom absorbent layer has a basis weight of about 102 grams per square meter and about 45% superabsorbent material.

80. The absorbent material in accordance with claim 78 in which said top absorbent layer and middle absorbent layer contain pulp which is 100% southern pine Kraft processed pulp.

81. The absorbent material in accordance with claim 78 in which said bottom absorbent layer includes southern pine pulp in a blend of 74% by weight of Kraft processed pulp and 16% by weight of cold caustic treated Kraft processed pulp.

82. The absorbent material in accordance with claim 63 in which said carrier layer comprises a layer of tissue produced from substantially 100% soft wood and having (a) a basis weight of between about 10 and about 13 pounds per 3,000 square feet, (b) a dry tensile strength in the machine direction of between about 275 and about 575 grams per inch, (c) a dry tensile strength in the cross direction of between about 60 and about 125 grams per inch, and (d) a porosity of between about 200 and about 312 cubic feet per minute per square foot.

83. The absorbent material in accordance with claim 63 in which said carrier layer comprises a first layer of tissue, and said web includes a second layer of tissue on a side of said web facing away from said first layer of tissue.

84. The absorbent material in accordance with claim 63 in which said machine direction tensile strength is greater than about 12 newtons.
